SEQUENCE LISTING (23 sequences)

SEQ ID NO: 1 - DNA, 291 bp, Bacillus licheniformis
  source 1..291: mol_type = genomic DNA
gtggaagttaccgacgtaagattacgccgcgtgaataccgatggtcgcatgagagcgattgcatccatcacgctggatcacgaatttgtagtgcatgatattcgtgtaattgatggaaacaatggtcttttcgttgcgatgccaagtaagcgtacacctgatggagaatttcgtgatatcgctcatccaatcaactcaagcacccgcggaaaaattcaggacgcggtgttaaatgaatatcatcgtttgggtgacgttgaggaaatagaatatgaagaagctggagcttct

SEQ ID NO: 2 - protein, 97 aa, Bacillus licheniformis
VEVTDVRLRRVNTDGRMRAIASITLDHEFVVHDIRVIDGNNGLFVAMPSKRTPDGEFRDIAHPINSSTRGKIQDAVLNEYHRLGDVEEIEYEEAGAS

SEQ ID NO: 3 - DNA, 774 bp, synthetic construct
  misc_feature 1..774: note = artificial sequence, cutinase
  source 1..774: mol_type = other DNA
tcaaacccgtatcaaagaggaccgaatcccacaagatccgccttaacagcggacggaccattttctgtcgcgacctatactgtgagtcgtctttctgtgagcggatttggcggtggcgttatctactatccgaccgggacgagcctgacttttggagggattgcgatgtctccaggatacactgccgacgcctcttctttggcctggttaggaagacgcttggcttcccatgggttcgtggttttggtgatcaatacaaacagtagatttgatggaccggattcgcgtgcctcgcaactgtcagctgccctcaactacctcagaacttcctctccctccgctgtaagagcacgcttggatgccaacagattggccgtagcaggacattcaatgggaggaggcggtacgctccggattgcggaacaaaatccgtctttgaaagccgcagtaccgttgacgccgtggcacacagacaaaacctttaacacctccgttcccgtgttgatcattggagccgaagccgatacagttgcaccggtcagccaacatgctattccgttttaccagaacttaccgtctacaacaccgaaagtctacgtcgagctctgtaatgcatcccatattgcaccgaattcgaacaatgccgcaattagcgtgtacactatttcatggatgaaactctgggtggacaatgatacaagatatcggcagtttttgtgcaacgtaaatgaccctgcgctttgcgactttcggacaaacaaccgccattgccag

SEQ ID NO: 4 - protein, 258 aa, synthetic construct
  REGION 1..258: note = artificial sequence, cutinase
SNPYQRGPNPTRSALTADGPFSVATYTVSRLSVSGFGGGVIYYPTGTSLTFGGIAMSPGYTADASSLAWLGRRLASHGFVVLVINTNSRFDGPDSRASQLSAALNYLRTSSPSAVRARLDANRLAVAGHSMGGGGTLRIAEQNPSLKAAVPLTPWHTDKTFNTSVPVLIIGAEADTVAPVSQHAIPFYQNLPSTTPKVYVELCNASHIAPNSNNAAISVYTISWMKLWVDNDTRYRQFLCNVNDPALCDFRTNNRHCQ

SEQ ID NO: 5 - DNA, 375 bp, Bacillus licheniformis
  source 1..375: mol_type = genomic DNA
atgacaaaggttgttcagacaaaagaagcaccggcggcgatcggaccgtattcacaaggaattgttgtcaacaatatgttttacagttcagggcaaattccgctgaccccgtcgggtgaaatggtgactggagacatcacagaacagacgcaccaggttttccgcaatttaaaagccgtgctgaaagaagcgggcgcctctctggagactgtcgtcaaagcaaccgtgtttctggcggatatgaatcaattcacagaggtgaatgaagtttacggacagtacttcgatacccacaaaccggcgagatcatgcgttgaggtggcgaggctgccgaaagacgcgcttgttgaaatagaagtcatcgcattagtgaaa

SEQ ID NO: 6 - protein, 125 aa, Bacillus licheniformis
MTKVVQTKEAPAAIGPYSQGIVVNNMFYSSGQIPLTPSGEMVTGDITEQTHQVFRNLKAVLKEAGASLETVVKATVFLADMNQFTEVNEVYGQYFDTHKPARSCVEVARLPKDALVEIEVIALVK

SEQ ID NO: 7 - DNA, 457 bp, Bacillus licheniformis
  source 1..457: mol_type = genomic DNA
atggttttggtctcaaaatcatcttttttgtttggaaaatttgtcaattcagtgtttttttatcccaatattacaaaaatatttttaattatgcaggaaaacaaaaaaagccgttgaatagttacactaatgtttttatattgggaaaaggtggtgaactactgtggaagttaccgacgtaagattacgccgcgtgaataccgatggtcgcatgagagcgattgcatccatcacgctggatcacgaatttgtagtgcatgatattcgtgtaattgatggaaacaatggtcttttcgttgcgatgccaagtaagcgtacacctgatggagaatttcgtgatatcgctcatccaatcaactcaagcacccgcggaaaaattcaggacgcggtgttaaatgaatatcatcgtttgggtgacgttgaggaaatagaatatgaagaagctggagcttcttaa

SEQ ID NO: 8 - DNA, 693 bp, synthetic construct
  misc_feature 1..693: note = artificial sequence, yabJ-spoVG
  source 1..693: mol_type = other DNA
atgacaaaggttgttcagacaaaagaagcaccggcggcgatcggaccgtattcacaaggaattgttgtcaacaatatgttttacagttcagggcaaattccgctgaccccgtcgggtgaaatggtgactggagacatcacagaacagacgcaccaggttttccgcaatttaaaagccgtgctgaaagaagcgggcgcctctctggagactgtcgtcaaagcaaccgtgtttctggcggatatgaatcaattcacagaggtgaatgaagtttacggacagtacttcgatacccacaaaccggcgagatcatgcgttgaggtggcgaggctgccgaaagacgcgcttgttgaaatagaagtcatcgcattagtgaaataggggaaaaggtggtgaactactgtggaagttaccgacgtaagattacgccgcgtgaataccgatggtcgcatgagagcgattgcatccatcacgctggatcacgaatttgtagtgcatgatattcgtgtaattgatggaaacaatggtcttttcgttgcgatgccaagtaagcgtacacctgatggagaatttcgtgatatcgctcatccaatcaactcaagcacccgcggaaaaattcaggacgcggtgttaaatgaatatcatcgtttgggtgacgttgaggaaatagaatatgaagaagctggagcttcttaa

SEQ ID NO: 9 - DNA, 851 bp, Bacillus licheniformis
  source 1..851: mol_type = genomic DNA
atgacaaaggttgttcagacaaaagaagcaccggcggcgatcggaccgtattcacaaggaattgttgtcaacaatatgttttacagttcagggcaaattccgctgaccccgtcgggtgaaatggtgactggagacatcacagaacagacgcaccaggttttccgcaatttaaaagccgtgctgaaagaagcgggcgcctctctggagactgtcgtcaaagcaaccgtgtttctggcggatatgaatcaattcacagaggtgaatgaagtttacggacagtacttcgatacccacaaaccggcgagatcatgcgttgaggtggcgaggctgccgaaagacgcgcttgttgaaatagaagtcatcgcattagtgaaatagaaagaactcttaaaagatggttttggtctcaaaatcatcttttttgtttggaaaatttgtcaattcagtgtttttttatcccaatattacaaaaatatttttaattatgcaggaaaacaaaaaaagccgttgaatagttacactaatgtttttatattgggaaaaggtggtgaactactgtggaagttaccgacgtaagattacgccgcgtgaataccgatggtcgcatgagagcgattgcatccatcacgctggatcacgaatttgtagtgcatgatattcgtgtaattgatggaaacaatggtcttttcgttgcgatgccaagtaagcgtacacctgatggagaatttcgtgatatcgctcatccaatcaactcaagcacccgcggaaaaattcaggacgcggtgttaaatgaatatcatcgtttgggtgacgttgaggaaatagaatatgaagaagctggagcttcttaa

SEQ ID NO: 10 - DNA, 511 bp, Bacillus licheniformis
  source 1..511: mol_type = genomic DNA
atgacaaaggttgttcagacaaaagaagcaccggcggcgatcggaccgtattcacaaggaattgttgtcaacaatatgttttacagttcagggcaaattccgctgaccccgtcgggtgaaatggtgactggagacatcacagaacagacgcaccaggttttccgcaatttaaaagccgtgctgaaagaagcgggcgcctctctggagactgtcgtcaaagcaaccgtgtttctggcggatatgaatcaattcacagaggtgaatgaagtttacggacagtacttcgatacccacaaaccggcgagatcatgcgttgaggtggcgaggctgccgaaagacgcgcttgttgaaatagaagtcatcgcattagtgaaatagaaagaactcttaaaagatggttttggtctcaaaatcatcttttttgtttggaaaatttgtcaattcagtgtttttttatcccaatattacaaaaatatttttaattatgcaggaaaacaaaaaaagccgttga

SEQ ID NO: 11 - DNA, 1906 bp, synthetic construct
  misc_feature 1..1906: note = artificial sequence, SP32-cutinase cassette - yabJ-BLP00052-spoVG - TamyL-attP
  source 1..1906: mol_type = other DNA
atgacaatgaaaaaaggcctaatgatcagcctgctcattttttcgctgattgtttcgttgtttgcgccagttggatctgcggatgcgtcaaacccgtatcaaagaggaccgaatcccacaagatccgccttaacagcggacggaccattttctgtcgcgacctatactgtgagtcgtctttctgtgagcggatttggcggtggcgttatctactatccgaccgggacgagcctgacttttggagggattgcgatgtctccaggatacactgccgacgcctcttctttggcctggttaggaagacgcttggcttcccatgggttcgtggttttggtgatcaatacaaacagtagatttgatggaccggattcgcgtgcctcgcaactgtcagctgccctcaactacctcagaacttcctctccctccgctgtaagagcacgcttggatgccaacagattggccgtagcaggacattcaatgggaggaggcggtacgctccggattgcggaacaaaatccgtctttgaaagccgcagtaccgttgacgccgtggcacacagacaaaacctttaacacctccgttcccgtgttgatcattggagccgaagccgatacagttgcaccggtcagccaacatgctattccgttttaccagaacttaccgtctacaacaccgaaagtctacgtcgagctctgtaatgcatcccatattgcaccgaattcgaacaatgccgcaattagcgtgtacactatttcatggatgaaactctgggtggacaatgatacaagatatcggcagtttttgtgcaacgtaaatgaccctgcgctttgcgactttcggacaaacaaccgccattgccagtagtaggatccagaagatgaataggaaatggggagtttgagatgacaaaggttgttcagacaaaagaagcaccggcggcgatcggaccgtattcacaaggaattgttgtcaacaatatgttttacagttcagggcaaattccgctgaccccgtcgggtgaaatggtgactggagacatcacagaacagacgcaccaggttttccgcaatttaaaagccgtgctgaaagaagcgggcgcctctctggagactgtcgtcaaagcaaccgtgtttctggcggatatgaatcaattcacagaggtgaatgaagtttacggacagtacttcgatacccacaaaccggcgagatcatgcgttgaggtggcgaggctgccgaaagacgcgcttgttgaaatagaagtcatcgcattagtgaaatagaaagaactcttaaaagatggttttggtctcaaaatcatcttttttgtttggaaaatttgtcaattcagtgtttttttatcccaatattacaaaaatatttttaattatgcaggaaaacaaaaaaagccgttgaatagttacactaatgtttttatattgggaaaaggtggtgaactactgtggaagttaccgacgtaagattacgccgcgtgaataccgatggtcgcatgagagcgattgcatccatcacgctggatcacgaatttgtagtgcatgatattcgtgtaattgatggaaacaatggtcttttcgttgcgatgccaagtaagcgtacacctgatggagaatttcgtgatatcgctcatccaatcaactcaagcacccgcggaaaaattcaggacgcggtgttaaatgaatatcatcgtttgggtgacgttgaggaaatagaatatgaagaagctggagcttcttaaccgcgtgctagcggccgcgtcgactagaagagcagagaggacggatttcctgaaggaaatccgtttttttattttgcccgtcttataaatttcgttgtccaactcgcttaattgcgagtttttatttcgtttatttcaattaaggtaactaaa

SEQ ID NO: 12 - DNA, 1819 bp, synthetic construct
  misc_feature 1..1819: note = artificial sequence, cutinase cassette + yabJ-BLP00052-spoVG - TamyL-attP
  source 1..1819: mol_type = other DNA
tcaaacccgtatcaaagaggaccgaatcccacaagatccgccttaacagcggacggaccattttctgtcgcgacctatactgtgagtcgtctttctgtgagcggatttggcggtggcgttatctactatccgaccgggacgagcctgacttttggagggattgcgatgtctccaggatacactgccgacgcctcttctttggcctggttaggaagacgcttggcttcccatgggttcgtggttttggtgatcaatacaaacagtagatttgatggaccggattcgcgtgcctcgcaactgtcagctgccctcaactacctcagaacttcctctccctccgctgtaagagcacgcttggatgccaacagattggccgtagcaggacattcaatgggaggaggcggtacgctccggattgcggaacaaaatccgtctttgaaagccgcagtaccgttgacgccgtggcacacagacaaaacctttaacacctccgttcccgtgttgatcattggagccgaagccgatacagttgcaccggtcagccaacatgctattccgttttaccagaacttaccgtctacaacaccgaaagtctacgtcgagctctgtaatgcatcccatattgcaccgaattcgaacaatgccgcaattagcgtgtacactatttcatggatgaaactctgggtggacaatgatacaagatatcggcagtttttgtgcaacgtaaatgaccctgcgctttgcgactttcggacaaacaaccgccattgccagtagtaggatccagaagatgaataggaaatggggagtttgagatgacaaaggttgttcagacaaaagaagcaccggcggcgatcggaccgtattcacaaggaattgttgtcaacaatatgttttacagttcagggcaaattccgctgaccccgtcgggtgaaatggtgactggagacatcacagaacagacgcaccaggttttccgcaatttaaaagccgtgctgaaagaagcgggcgcctctctggagactgtcgtcaaagcaaccgtgtttctggcggatatgaatcaattcacagaggtgaatgaagtttacggacagtacttcgatacccacaaaccggcgagatcatgcgttgaggtggcgaggctgccgaaagacgcgcttgttgaaatagaagtcatcgcattagtgaaatagaaagaactcttaaaagatggttttggtctcaaaatcatcttttttgtttggaaaatttgtcaattcagtgtttttttatcccaatattacaaaaatatttttaattatgcaggaaaacaaaaaaagccgttgaatagttacactaatgtttttatattgggaaaaggtggtgaactactgtggaagttaccgacgtaagattacgccgcgtgaataccgatggtcgcatgagagcgattgcatccatcacgctggatcacgaatttgtagtgcatgatattcgtgtaattgatggaaacaatggtcttttcgttgcgatgccaagtaagcgtacacctgatggagaatttcgtgatatcgctcatccaatcaactcaagcacccgcggaaaaattcaggacgcggtgttaaatgaatatcatcgtttgggtgacgttgaggaaatagaatatgaagaagctggagcttcttaaccgcgtgctagcggccgcgtcgactagaagagcagagaggacggatttcctgaaggaaatccgtttttttattttgcccgtcttataaatttcgttgtccaactcgcttaattgcgagtttttatttcgtttatttcaattaaggtaactaaa

SEQ ID NO: 13 - DNA, 2794 bp, synthetic construct
  misc_feature 1..2794: note = artificial sequence, triple promoter-SP32-cutinase cassette + yabJ-BLP00052-spoVG - TamyL-attP
  source 1..2794: mol_type = other DNA
ataaatgagtagaaagcgccatatcggcgcttttcttttggaagaaaatatagggaaaatggtacttgttaaaaattcggaatatttatacaatatcatatgtatcacattgaaaggaggggcctgctgtccagactgtccgctgtgtaaaaaaaaggaataaaggggggttgacattattttactgatatgtataatataatttgtataagaaaatggaggggccctcgaaacgtaagatgaaaccttagataaaagtgctttttttgttgcaattgaagaattattaatgttaagcttaattaaagataatatctttgaattgtaacccccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatagaaaggaggtgatccagccgcaccttatgaaaaatcattttatcagtttgaaaattatgtattatggagctcagaggaggagagtgagtaatgacaatgaaaaaaggcctaatgatcagcctgctcattttttcgctgattgtttcgttgtttgcgccagttggatctgcggatgcgtcaaacccgtatcaaagaggaccgaatcccacaagatccgccttaacagcggacggaccattttctgtcgcgacctatactgtgagtcgtctttctgtgagcggatttggcggtggcgttatctactatccgaccgggacgagcctgacttttggagggattgcgatgtctccaggatacactgccgacgcctcttctttggcctggttaggaagacgcttggcttcccatgggttcgtggttttggtgatcaatacaaacagtagatttgatggaccggattcgcgtgcctcgcaactgtcagctgccctcaactacctcagaacttcctctccctccgctgtaagagcacgcttggatgccaacagattggccgtagcaggacattcaatgggaggaggcggtacgctccggattgcggaacaaaatccgtctttgaaagccgcagtaccgttgacgccgtggcacacagacaaaacctttaacacctccgttcccgtgttgatcattggagccgaagccgatacagttgcaccggtcagccaacatgctattccgttttaccagaacttaccgtctacaacaccgaaagtctacgtcgagctctgtaatgcatcccatattgcaccgaattcgaacaatgccgcaattagcgtgtacactatttcatggatgaaactctgggtggacaatgatacaagatatcggcagtttttgtgcaacgtaaatgaccctgcgctttgcgactttcggacaaacaaccgccattgccagtagtaggatccagaagatgaataggaaatggggagtttgagatgacaaaggttgttcagacaaaagaagcaccggcggcgatcggaccgtattcacaaggaattgttgtcaacaatatgttttacagttcagggcaaattccgctgaccccgtcgggtgaaatggtgactggagacatcacagaacagacgcaccaggttttccgcaatttaaaagccgtgctgaaagaagcgggcgcctctctggagactgtcgtcaaagcaaccgtgtttctggcggatatgaatcaattcacagaggtgaatgaagtttacggacagtacttcgatacccacaaaccggcgagatcatgcgttgaggtggcgaggctgccgaaagacgcgcttgttgaaatagaagtcatcgcattagtgaaatagaaagaactcttaaaagatggttttggtctcaaaatcatcttttttgtttggaaaatttgtcaattcagtgtttttttatcccaatattacaaaaatatttttaattatgcaggaaaacaaaaaaagccgttgaatagttacactaatgtttttatattgggaaaaggtggtgaactactgtggaagttaccgacgtaagattacgccgcgtgaataccgatggtcgcatgagagcgattgcatccatcacgctggatcacgaatttgtagtgcatgatattcgtgtaattgatggaaacaatggtcttttcgttgcgatgccaagtaagcgtacacctgatggagaatttcgtgatatcgctcatccaatcaactcaagcacccgcggaaaaattcaggacgcggtgttaaatgaatatcatcgtttgggtgacgttgaggaaatagaatatgaagaagctggagcttcttaaccgcgtgctagcggccgcgtcgactagaagagcagagaggacggatttcctgaaggaaatccgtttttttattttgcccgtcttataaatttcgttgtccaactcgcttaattgcgagtttttatttcgtttatttcaatcaaggtaaatg

SEQ ID NO: 14 - DNA, 309 bp, synthetic construct
  misc_feature 1..309: note = artificial sequence, triple promoter
  source 1..309: mol_type = other DNA
ataaatgagtagaaagcgccatatcggcgcttttcttttggaagaaaatatagggaaaatggtacttgttaaaaattcggaatatttatacaatatcatatgtatcacattgaaaggaggggcctgctgtccagactgtccgctgtgtaaaaaaaaggaataaaggggggttgacattattttactgatatgtataatataatttgtataagaaaatggaggggccctcgaaacgtaagatgaaaccttagataaaagtgctttttttgttgcaattgaagaattattaatgttaagcttaattaaaga

SEQ ID NO: 15 - DNA, 87 bp, Bacillus pumilus
  source 1..87: mol_type = genomic DNA
atgacaatgaaaaaaggcctaatgatcagcctgctcattttttcgctgattgtttcgttgtttgcgccagttggatctgcggatgcg

SEQ ID NO: 16 - protein, 29 aa, Bacillus pumilus
MTMKKGLMISLLIFSLIVSLFAPVGSADA

SEQ ID NO: 17 - DNA, 309 bp, synthetic construct; identical to SEQ ID NO: 14
  misc_feature 1..309: note = artificial sequence, TamyL coding sequence
  source 1..309: mol_type = other DNA

SEQ ID NO: 18 - DNA, 557 bp, synthetic construct
  misc_feature 1..557: note = artificial sequence, cryIIIA mRNA stabilizer region
  source 1..557: mol_type = other DNA
taatatctttgaattgtaacgcccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatagaaaggaggtgatccagccgcaccttatgaaaaatcattttatcagtttgaaaattatgtattatg

SEQ ID NO: 19 - DNA, 294 bp, Bacillus subtilis
  source 1..294: mol_type = genomic DNA
gtggaagttactgacgtaagattacgccgcgtgaataccgatggtcgcatgagagcgattgcatccatcacgctggatcacgaatttgttgtacatgatattcgtgtgattgatggaaacaatggtcttttcgttgcgatgccgagtaaacgcacccctgatggagagttccgcgatattactcatcctattaattcaagcacgcgaggaaagattcaagatgccgtgttaaatgagtatcatcgtctgggtgacactgaagcattagaattcgaagaagctggagcttcttaa

SEQ ID NO: 20 - protein, 97 aa, Bacillus subtilis
MEVTDVRLRRVNTDGRMRAIASITLDHEFVVHDIRVIDGNNGLFVAMPSKRTPDGEFRDITHPINSSTRGKIQDAVLNEYHRLGDTEALEFEEAGAS

SEQ ID NO: 21 - DNA, 1798 bp, synthetic construct
  misc_feature 1..1798: note = artificial sequence, triple promoter-yabJ-spoVG
  source 1..1798: mol_type = other DNA
ataaatgagtagaaagcgccatatcggcgcttttcttttggaagaaaatatagggaaaatggtacttgttaaaaattcggaatatttatacaatatcatatgtatcacattgaaaggaggggcctgctgtccagactgtccgctgtgtaaaaaaaaggaataaaggggggttgacattattttactgatatgtataatataatttgtataagaaaatggaggggccctcgaaacgtaagatgaaaccttagataaaagtgctttttttgttgcaattgaagaattattaatgttaagcttaattaaagataatatctttgaattgtaacgcccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatagaaaggaggtgatccagccgcaccttatgaaaaatcattttatcagtttgaaaattatgtattatggaagagctgaaaggggaggagaaccatggaactagtctggatcctacgcgttcatatgtatcacattgaaaggggaggagaatcatgacaaaggttgttcagacaaaagaagcaccggcggcgatcggaccgtattcacaaggaattgttgtcaacaatatgttttacagttcagggcaaattccgctgaccccgtcgggtgaaatggtgactggagacatcacagaacagacgcaccaggttttccgcaatttaaaagccgtgctgaaagaagcgggcgcctctctggagactgtcgtcaaagcaaccgtgtttctggcggatatgaatcaattcacagaggtgaatgaagtttacggacagtacttcgatacccacaaaccggcgagatcatgcgttgaggtggcgaggctgccgaaagacgcgcttgttgaaatagaagtcatcgcattagtgaaatagtgggaaaaggtggtgaactactgtggaagttaccgacgtaagattacgccgcgtgaataccgatggtcgcatgagagcgattgcatccatcacgctggatcacgaatttgtagtgcatgatattcgtgtaattgatggaaacaatggtcttttcgttgcgatgccaagtaagcgtacacctgatggagaatttcgtgatatcgctcatccaatcaactcaagcacccgcggaaaaattcaggacgcggtgttaaatgaatatcatcgtttgggtgacgttgaggaaatagaatatgaagaagctggagcttcttaatagccgcgtgctagcggccgcgtcgactagaagagcagagaggacggatttcctgaaggaaatccgtttttttattttgcccgtcttataaatttcgttgtccaactcgcttaattgcgagtttttatttcgtttatttcaatcaaggtaaatg

SEQ ID NO: 22 - DNA, 1644 bp, synthetic construct
  misc_feature 1..1644: note = artificial sequence, triple promoter-spoVG-yabJ
  source 1..1644: mol_type = other DNA
ataaatgagtagaaagcgccatatcggcgcttttcttttggaagaaaatatagggaaaatggtacttgttaaaaattcggaatatttatacaatatcatatgtatcacattgaaaggaggggcctgctgtccagactgtccgctgtgtaaaaaaaaggaataaaggggggttgacattattttactgatatgtataatataatttgtataagaaaatggaggggccctcgaaacgtaagatgaaaccttagataaaagtgctttttttgttgcaattgaagaattattaatgttaagcttaattaaagataatatctttgaattgtaacgcccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatagaaaggaggtgatccagccgcaccttatgaaaaatcattttatcagtttgaaaattatgtattatggaagagctgaaaggggaggagaaccatggaactagtctggatcctacgcgttcatatgtatcacattgaaaggggaggagaatctgtggaagttaccgacgtaagattacgccgcgtgaataccgatggtcgcatgagagcgattgcatccatcacgctggatcacgaatttgtagtgcatgatattcgtgtaattgatggaaacaatggtcttttcgttgcgatgccaagtaagcgtacacctgatggagaatttcgtgatatcgctcatccaatcaactcaagcacccgcggaaaaattcaggacgcggtgttaaatgaatatcatcgtttgggtgacgttgaggaaatagaatatgaagaagctggagcttcttaagggaaaaggtggtgaactactatgacaaaggttgttcagacaaaagaagcaccggcggcgatcggaccgtattcacaaggaattgttgtcaacaatatgttttacagttcagggcaaattccgctgaccccgtcgggtgaaatggtgactggagacatcacagaacagacgcaccaggttttccgcaatttaaaagccgtgctgaaagaagcgggcgcctctctggagactgtcgtcaaagcaaccgtgtttctggcggatatgaatcaattcacagaggtgaatgaagtttacggacagtacttcgatacccacaaaccggcgagatcatgcgttgaggtggcgaggctgccgaaagacgcgcttgttgaaatagaagtcatcgcattagtgaaatag

SEQ ID NO: 23 - DNA, 1245 bp, synthetic construct
  misc_feature 1..1245: note = artificial sequence, triple promoter-spoVG
  source 1..1245: mol_type = other DNA
ataaatgagtagaaagcgccatatcggcgcttttcttttggaagaaaatatagggaaaatggtacttgttaaaaattcggaatatttatacaatatcatatgtatcacattgaaaggaggggcctgctgtccagactgtccgctgtgtaaaaaaaaggaataaaggggggttgacattattttactgatatgtataatataatttgtataagaaaatggaggggccctcgaaacgtaagatgaaaccttagataaaagtgctttttttgttgcaattgaagaattattaatgttaagcttaattaaagataatatctttgaattgtaacgcccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatagaaaggaggtgatccagccgcaccttatgaaaaatcattttatcagtttgaaaattatgtattatggaagagctgaaaggggaggagaaccatggaactagtctggatcctacgcgttcatatgtatcacattgaaaggggaggagaatctgtggaagttaccgacgtaagattacgccgcgtgaataccgatggtcgcatgagagcgattgcatccatcacgctggatcacgaatttgtagtgcatgatattcgtgtaattgatggaaacaatggtcttttcgttgcgatgccaagtaagcgtacacctgatggagaatttcgtgatatcgctcatccaatcaactcaagcacccgcggaaaaattcaggacgcggtgttaaatgaatatcatcgtttgggtgacgttgaggaaatagaatatgaagaagctggagcttcttaa

(19) United States
(12) Patent Application Publication
(10) Pub. No.: US 2025/0034535 A1
(43) Pub. Date: Jan. 30, 2025

(54) IMPROVED PROTEIN PRODUCTION IN RECOMBINANT BACTERIA

(71) Applicant: Novozymes A/S, Bagsvaerd (DK)

(72) Inventors: Allan Kent NIELSEN, Copenhagen (DK); Rasmus Lund ANDERSEN, Vintrie (SE); Karen Fuglede APPEL, Soeborg (DK); Anna Lund VUKMAN, Copenhagen (DK)

(73) Assignee: Novozymes A/S, Bagsvaerd (DK)

(21) Appl. No.: 18/716,115

(22) PCT Filed: Dec. 7, 2022
(86) PCT No.: PCT/EP2022/084700

§ 371 Date: Jun. 3, 2024

(30) Foreign Application Priority Data
Dec. 10, 2021 (DK) PA202101180

(51) Int. Cl. C12N 9/18; C07K 14/32; C12N 1/20; C12N 15/75; C12R 1/10; C12R 1/125

(52) U.S. Cl. CPC C12N 9/18; C07K 14/32; C12N 1/20; C12N 15/75; C12R 2001/10; C12R 2001/125; C12Y 301/01074

(57) ABSTRACT

The present invention relates to mutated bacterial host cells with increased SpoVG polypeptide expression, and nucleic acid constructs and expression vectors encoding SpoVG polypeptides. The invention also relates to methods of producing a protein of interest using the mutated bacterial host cells.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to mutated bacterial host cells with increased SpoVG polypeptide expression, and nucleic acid constructs and expression vectors encoding SpoVG polypeptides. The invention also relates to methods of producing a protein of interest using the mutated bacterial host cells.

### Description of the Related Art

Recombinant gene expression in recombinant host cells, such as bacterial host cells, is a common method for recombinant protein production. Recombinant proteins produced in prokaryotic systems are enzymes and other valuable proteins. In industrial and commercial purposes, the productivity of the applied cell systems, i.e., the production of total protein per fermentation unit, is an important factor of production costs. Traditionally, yield increases have been achieved through mutagenesis and screening for increased production of proteins of interest. However, this approach is mainly only useful for the overproduction of endogenous proteins in isolates containing the enzymes of interest. Therefore, for each new protein or enzyme product, a lengthy strain and process development program is required to achieve improved productivities.

For the overexpression of heterologous proteins in prokaryotic systems, the production process is recognized as a complex multi-phase and multi-component process. Cell growth and product formation are determined by a wide range of parameters, including the composition of the culture medium, fermentation pH, fermentation temperature, dissolved oxygen tension, shear stress, and bacterial morphology.

Various approaches to improve transcription have been used in bacteria. For the expression of heterologous genes, codon-optimized, synthetic genes can improve the transcription rate (WO9923211, Novozymes A/S). To obtain high-level expression of a particular gene, a well-established procedure is targeting multiple copies of the recombinant gene constructs to the locus of a highly expressed endogenous gene. However, multi-copy strains often reach the host cell's expression limits, whereafter integration of additional copies of the recombinant gene does not further improve recombinant yields. Despite the presented approaches, it is of continuous interest to further improve recombinant protein production in bacterial host cells.

The object of the present invention is to provide a modified bacterial host strain and a method of protein production with increased productivity and/or yield of recombinant protein.

### SUMMARY OF THE INVENTION

As disclosed herein, the inventors of the present invention have identified that for increasing yields during recombinant protein production, increasing the copy number of the gene encoding the recombinant protein is not necessarily leading to recombinant protein yield improvements. Surprisingly, the inventors have shown that in bacterial cells producing a recombinant protein of interest, overexpression of a gene encoding a SpoVG polypeptide improves secretion and/or yield of the recombinant protein of interest. After overexpression of the spoVG gene, recombinant protein yield was improved significantly, i.e., by 9-18%, when compared with protein yield from host cells comprising only a native spoVG gene. This surprising effect was shown in host cells comprising different copy numbers of the gene encoding the protein of interest.

In a first aspect, the present invention relates to a recombinant bacterial host cell comprising in its genome at least one first heterologous promoter operably linked to at least one first polynucleotide encoding a stage 5 sporulation protein G (SpoVG) polypeptide, a SpoVG fragment, or a SpoVG variant.

In a second aspect, the present invention relates to method for producing one or more polypeptide of interest, the method comprising:
i) providing a bacterial host cell according to the first aspect,
ii) cultivating said host cell under conditions conducive for expression of the one or more polypeptide of interest; and,
iii) optionally recovering the one or more polypeptide of interest.

In a third aspect, the present invention relates to a nucleic acid construct comprising at least one first heterologous promoter operably linked to at least one first polynucleotide encoding a stage 5 sporulation protein G (SpoVG) polypeptide, a SpoVG fragment, or a SpoVG variant.

In a fourth aspect, the present invention relates to an expression vector comprising a nucleic acid construct according to the third aspect.

In a fifth aspect, the present invention relates to a method for production of a recombinant bacterial host cell with increased expression of a polypeptide of interest, the method comprising:
i) providing a parent bacterial host cell comprising in its genome a native polynucleotide encoding a SpoVG polypeptide, SpoVG variant, or SpoVG fragment, and a second polynucleotide encoding a polypeptide of interest, and
ii) introducing into said parent bacterial host cell a first polynucleotide encoding the SpoVG polypeptide, fragment or variant,
wherein the recombinant bacterial host cell comprises increased expression of the polypeptide of interest and the SpoVG polypeptide, fragment or variant relative to the parent bacterial host cell when cultivated under identical conditions.

### BRIEF DESCRIPTION OF THE FIGURES

FIG. 1 shows a schematic map of plasmid pCLK015.

FIG. 2 shows fed-batch cultivation of strains expressing recombinant cutinase (relative cutinase activity plotted against cultivation time [h]).

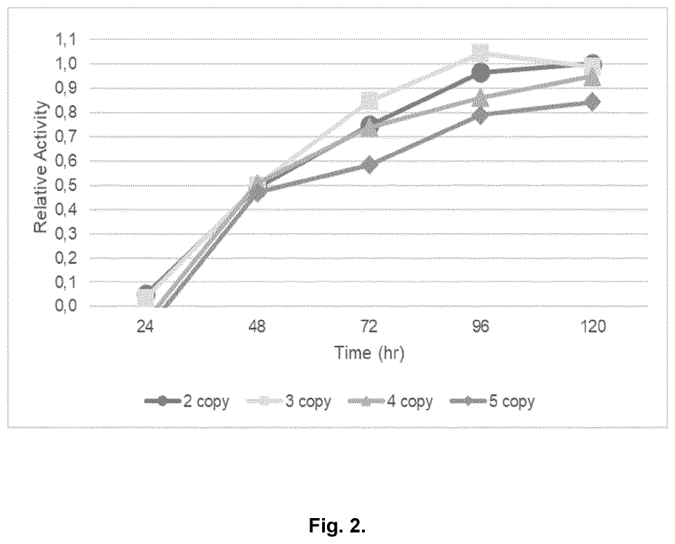

FIG. 3 shows a schematic overview of the yabJ-spoVG operon.

FIG. 4 shows a schematic map of pBT14199.

FIG. 5 shows fed-batch cultivation of strains expressing recombinant cutinase (relative cutinase activity plotted against cultivation time [h]).

### DEFINITIONS

In accordance with this detailed description, the following definitions apply. Note that the singular forms “a,” “an,” and “the” include plural references unless the context clearly dictates otherwise.

Unless defined otherwise or clearly indicated by context, all technical and scientific terms used herein have the same meaning as commonly understood by one of ordinary skill in the art to which this invention belongs. cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon, such as ATG, GTG, or TTG, and ends with a stop codon, such as TAA, TAG, or TGA. The coding sequence may be a genomic DNA, cDNA, synthetic DNA, or a combination thereof.

Control sequences: The term “control sequences” means nucleic acid sequences involved in regulation of expression of a polynucleotide in a specific organism or in vitro. Each control sequence may be native (i.e., from the same gene) or heterologous (i.e., from a different gene) to the polynucleotide encoding the polypeptide, and native or heterologous to each other. Such control sequences include, but are not limited to leader, polyadenylation, prepropeptide, propeptide, signal peptide, promoter, terminator, enhancer, and transcription or translation initiator and terminator sequences. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Cutinase: The term “cutinase” means a polypeptide having cutinase activity (EC 3.1.1.74), such as polyethylene terephthalate (PET) hydrolase activity, that catalyzes the hydrolysis of cutin and/or the hydrolysis of p-nitrophenyl esters of hexadecenoic acid. For purposes of the present invention, cutinase activity, i.e. PET hydrolase activity, may be determined according to the procedures described in the Examples.

Downstream/Upstream: The term “downstream” or “at its 3′ end” means that a particular polynucleotide sequence, e.g. sequence 2, is located at the 3′ end of the coding strand of a genetic locus or gene sequence, e.g. sequence 1, so that the orientation on the coding strand is: 5′-sequence 1→sequence 2-3′. The term “upstream” or “at its 5′ end” means that a particular polynucleotide sequence, e.g. sequence 2, is located at the 5′ end of the coding strand of a genetic locus or gene sequence, e.g. sequence 1, so that the orientation on the coding strand is: 5′-sequence 2→sequence 1-3′.

Endogenous: The term “endogenous” means, with respect to a host cell, that a polypeptide or nucleic acid does naturally occur in the host cell, i.e., the polypeptide or nucleic acid is from a gene naturally comprised in the host cell.

Exogenous: The term “exogenous” means, with respect to a host cell, that a polypeptide or nucleic acid does not naturally occur in the host cell, i.e., the polypeptide or nucleic acid is from a gene not comprised in the host cell, but instead derived from another organism than the host cell.

Expression: The term “expression” means any step involved in the production of a polypeptide including, but not limited to, transcription, post-transcriptional modification, translation, post-translational modification, and secretion.

Expression vector: An “expression vector” refers to a linear or circular DNA construct comprising a DNA sequence encoding a polypeptide, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

Extension: The term “extension” means an addition of one or more amino acids to the amino and/or carboxyl terminus of a SpoVG polypeptide, wherein the “extended” SpoVG polypeptide has SpoVG activity and achieves a similar/identical function relative to the non-extended SpoVG polypeptide, leading to increased product yield after overexpression of the extended SpoVG polypeptide.

Fragment: The term “fragment” means a polypeptide having one or more amino acids absent from the amino and/or carboxyl terminus of the mature SpoVG polypeptide, wherein the SpoVG fragment has SpoVG activity and achieves a similar/identical function relative to the non-extended SpoVG polypeptide, leading to increased product yield after overexpression of the SpoVG fragment.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, of a polypeptide or nucleic acid is not naturally associated with the polypeptide or nucleic acid, i.e., the control sequence is from a gene other than the gene encoding the mature polypeptide.

Host Strain or Host Cell: A “host strain” or “host cell” is an organism into which an expression vector, phage, virus, or other DNA construct, including a polynucleotide encoding a polypeptide of interest (e.g., an amylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

Introduced: The term “introduced” in the context of inserting a nucleic acid sequence into a cell, means “transfection”, “transformation” or “transduction,” as known in the art.

Isogenic: The term “isogenic” refers to a host cell or population of host cells with essentially identical genes. A parent host cell is considered to be isogenic to its daughter host cell when no mutations have been introduced into the genome of the daughter host cell. A parent host cell is considered “otherwise isogenic” to its daughter host cell, when only a specific, known set of one or more mutation(s) have been introduced into the daughter cell(s), but no further unknown or other mutations. A non-limiting example for an otherwise isogenic parent cell is when a daughter cell comprises an additional copy of a spoVG gene compared to the parental cell, but does not comprise any further mutations compared to the parental cell.

Isolated: The term “isolated” means a polypeptide, nucleic acid, cell, or other specified material or component that has been separated from at least one other material or component, including but not limited to, other proteins, nucleic acids, cells, etc. An isolated polypeptide, nucleic acid, cell or other material is thus in a form that does not occur in nature. An isolated polypeptide includes, but is not limited to, a culture broth containing the secreted polypeptide expressed in a host cell.

Mature polypeptide: The term “mature polypeptide” means a polypeptide in its mature form following N-terminal and/or C-terminal processing (e.g., removal of signal peptide). In one aspect, the mature polypeptide is SEQ ID NO: 2. In another aspect, the mature polypeptide is SEQ ID NO: 4.

Mature polypeptide coding sequence: The term “mature polypeptide coding sequence” means a polynucleotide that encodes a mature polypeptide having enzyme activity, such as cutinase activity, or such as SpoVG activity. In one aspect, the mature polypeptide coding sequence is nucleotides 1 to 97 of SEQ ID NO: 2. In another aspect, the mature polypeptide coding sequence is nucleotides 1 to 291 of SEQ ID NO: 1.

Native: The term “native” means a nucleic acid or polypeptide naturally occurring in a host cell. A non-limiting example of a native nucleic acid or native polypeptide for Bacillus licheniformis are the polynucleotide of SEQ ID NO: 1 and the polypeptide of SEQ ID NO: 2, resprectively.

Nucleic acid: The term “nucleic acid” encompasses DNA, RNA, heteroduplexes, and synthetic molecules capable of encoding a polypeptide. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, and which comprises one or more control sequences operably linked to the nucleic acid sequence.

Operably linked: The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequence.

Purified: The term “purified” means a nucleic acid, polypeptide or cell that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight or on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

In one aspect, the term “purified” as used herein refers to the polypeptide or cell being essentially free from components (especially insoluble components) from the production organism. In other aspects. the term “purified” refers to the polypeptide being essentially free of insoluble components (especially insoluble components) from the native organism from which it is obtained. In one aspect, the polypeptide is separated from some of the soluble components of the organism and culture medium from which it is recovered. The polypeptide may be purified (i.e., separated) by one or more of the unit operations filtration, precipitation, or chromatography.

Accordingly, the polypeptide may be purified such that only minor amounts of other proteins, in particular, other polypeptides, are present. The term “purified” as used herein may refer to removal of other components, particularly other proteins and most particularly other enzymes present in the cell of origin of the polypeptide. The polypeptide may be “substantially pure”, i.e., free from other components from the organism in which it is produced, e.g., a host organism for recombinantly produced polypeptide. In one aspect, the polypeptide is at least 40% pure by weight of the total polypeptide material present in the preparation. In one aspect, the polypeptide is at least 50%, 60%, 70%, 80% or 90% pure by weight of the total polypeptide material present in the preparation. As used herein. a “substantially pure polypeptide” may denote a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which the polypeptide is natively or recombinantly associated.

It is, therefore, preferred that the substantially pure polypeptide is at least 92% pure, preferably at least 94% pure, more preferably at least 95% pure, more preferably at least 96% pure, more preferably at least 97% pure, more preferably at least 98% pure, even more preferably at least 99% pure, most preferably at least 99.5% pure by weight of the total polypeptide material present in the preparation. The polypeptide of the present invention is preferably in a substantially pure form (i.e., the preparation is essentially free of other polypeptide material with which it is natively or recombinantly associated). This can be accomplished, for example by preparing the polypeptide by well-known recombinant methods or by classical purification methods.

Recombinant: The term “recombinant” is used in its conventional meaning to refer to the manipulation, e.g., cutting and rejoining, of nucleic acid sequences to form constellations different from those found in nature. The term recombinant refers to a cell, nucleic acid, polypeptide or vector that has been modified from its native state. Thus, for example, recombinant cells express genes that are not found within the native (non-recombinant) form of the cell, or express native genes at different levels or under different conditions than found in nature. The term “recombinant” is synonymous with “genetically modified” and “transgenic”.

Recover: The terms “recover” or “recovery” means the removal of a polypeptide from at least one fermentation broth component selected from the list of a cell, a nucleic acid, or other specified material, e.g., recovery of the polypeptide from the whole fermentation broth, or from the cell-free fermentation broth, by polypeptide crystal harvest, by filtration, e.g. depth filtration (by use of filter aids or packed filter medias, cloth filtration in chamber filters, rotary-drum filtration, drum filtration, rotary vacuum-drum filters, candle filters, horizontal leaf filters or similar, using sheed or pad filtration in framed or modular setups) or membrane filtration (using sheet filtration, module filtration, candle filtration, microfiltration, ultrafiltration in either cross flow, dynamic cross flow or dead end operation), or by centrifugation (using decanter centrifuges, disc stack centrifuges, hyrdo cyclones or similar), or by precipitating the polypeptide and using relevant solid-liquid separation methods to harvest the polypeptide from the broth media by use of classification separation by particle sizes. Recovery encompasses isolation and/or purification of the polypeptide.

Sequence identity: The relatedness between two amino acid sequences or between two nucleotide sequences is described by the parameter “sequence identity”. Sequence identity is determined by the following method:

The sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), version 6.6.0. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

The sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), version 6.6.0. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

Signal Peptide: A “signal peptide” is a sequence of amino acids attached to the N-terminal portion of a protein, which facilitates the secretion of the protein outside the cell. The mature form of an extracellular protein lacks the signal peptide, which is cleaved off during the secretion process.

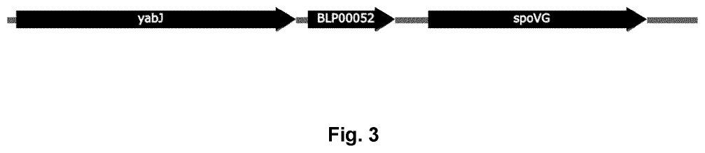

SpoVG: The term “SpoVG” means a stage V sporulation protein G. The name derives from observations that Bacillus ssp. spoVG mutants are unable to complete stage five of sporulation (Matsuno K, and Sonenshein AL, J. Bacteriol., 1999, vol. 181, pg. 3392-3401). However, in non-sporulating bacteria, its mode of action and the molecular mechanisms involved remain to be investigated in more details. It was shown that SpoVG is a site-specific DNA-binding protein, and that in the highly conserved SpoVG family, a six amino acid residue stretch of the SpoVG alpha-helix contributes to DNA sequence specificity (amino acids corresponding to amino acids at positions 66-71 of SEQ ID NO: 2), and two highly conserved, positively charged amino acid residues on an adjacent beta-sheet (amino acids corresponding to amino acids at positions 50-51 of SEQ ID NO: 2) are essential for DNA-binding, apparently by contacts with the DNA phosphate backbone (Jutras et al., 2013, PLos ONE 8(6): e66683. doi:101371/journal.pone.0066683). In wildtype cells, SpoVG is encoded on the yabJ-spoVG operon comprising from 5′ to 3′: a YabJ coding sequence, a putative ORF BLP00052, and a SpoVG coding sequence, as depicted in FIG. 3.

SpoVG activity: The term “SpoVG activity” means a site-specific DNA-binding activity related to the six amino acid residue stretch of the SpoVG alpha-helix (amino acids corresponding to amino acids at positions 66-71 of SEQ ID NO: 2), and the two highly conserved, positively charged amino acid residues on an adjacent beta-sheet (amino acids corresponding to amino acids at positions 50-51 of SEQ ID NO: 2) which are essential for DNA-binding. Additionally or alternatively, SpoVG activity means site-specific RNA-binding activity and/or polypeptide-binding activity. By providing an additional spoVG gene copy, the inventors successfully increased overall SpoVG activity which surprisingly resulted in increased protein yield during the expression of recombinant protein. In one aspect of the invention, increased SpoVG activity is thus associated with increased recombinant protein yield. DNA-binding activity can be determined according to the methods provided in Jutras et al., 2013, supra.

Subsequence: The term “subsequence” means a polynucleotide having one or more nucleotides absent from the 5′ and/or 3′ end of a mature polypeptide coding sequence; wherein the subsequence encodes a fragment having SpoVG activity.

Variant: The term “variant” means a polypeptide having SpoVG activity comprising a man-made mutation, i.e., a substitution, insertion (including extension), and/or deletion (e.g., truncation), at one or more positions. A substitution means replacement of the amino acid occupying a position with a different amino acid; a deletion means removal of the amino acid occupying a position; and an insertion means adding 1-5 amino acids (e.g., 1-3 amino acids, in particular, 1 amino acid) adjacent to and immediately following the amino acid occupying a position.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

YabJ: The term “YabJ” means a YabJ polypeptide encoded by the yabJ-spoVG operon in wildtype cells. YabJ belongs to the YjgF protein family of unknown biochemical function. Although YabJ and SpoVG are polypeptides encoded by the same operon, both polypeptides are expressed as single YabJ and SpoVG polypeptides, respectively, i.e. not being comprised in the same polypeptide chain.

### DETAILED DESCRIPTION OF THE INVENTION

### Host Cells

In a first aspect, the present invention relates to a recombinant bacterial host cell comprising in its genome at least one first heterologous promoter operably linked to at least one first polynucleotide encoding a stage 5 sporulation protein G (SpoVG) polypeptide, a SpoVG fragment, or a SpoVG variant. Additionally or alternatively to increasing spoVG gene copy number, SpoVG expression can be increased by replacing its promoter with a stronger (synthetic) promoter, by CRISPR-activation technologies, by RNAi or by any other suitable methods known to the skilled person in the art.

In one embodiment, the SpoVG polypeptide comprises a protein-binding activity.

In one embodiment, the SpoVG polypeptide comprises a RNA-binding activity.

In a particular embodiment, the SpoVG polypeptide comprises a DNA-binding activity. Preferably the DNA-binding activity is a site-specific DNA-binding activity related to the six amino acid residue stretch of the SpoVG alpha-helix (amino acids corresponding to amino acids at positions 66-71 of SEQ ID NO: 2, “SSTRGK”). Additionally or alternatively, the DNA-binding activity is related to two highly conserved, positively charged amino acid residues on a beta-sheet (amino acids corresponding to amino acids at positions 50-51 of SEQ ID NO: 2, “KR”).

In one embodiment, the SpoVG polypeptide is a SpoVG fragment comprising at least the amino acids corresponding to amino acids 50-71 of SEQ ID NO: 2, “KRTPDGEFRDIAHPINSSTRGK”.

In one embodiment, the SpoVG polypeptide is a SpoVG fragment or SpoVG variant comprising an polypeptide which comprises or consists of a amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of positions 50-71 of SEQ ID NO: 2, “KRTPDGEFRDIAHPINSSTRGK”.

By providing an additional spoVG gene copy, the inventors successfully increased overall SpoVG activity which surprisingly resulted in increased protein yield during the expression of recombinant protein. In one aspect of the invention, increased SpoVG activity is thus associated with increased recombinant protein yield.

In one embodiment, the host cell comprises a mutation in its native yabJ/spoVG operon, selected from the list of a polynucleotide deletion, a polynucleotide insertion and a polynucleotide substitution.

In one embodiment, the host cell with the mutation in the native yabJ/spoVG operon encodes a mutated YabJ polypeptide, fragment or variant, and/or a SpoVG polypeptide, fragment or variant, comprising at least one amino acid deletion, amino acid insertion, and/or amino acid substitution.

In one embodiment, the host cell is further comprising in its genome at least one second polynucleotide encoding at least one polypeptide of interest. The polypeptide of interest may be endogenous to the host cell, or exogenous to the host cell.

In one embodiment, the second polynucleotide is operably linked a heterologous promoter. Preferably, the second polynucleotide is operably linked to the first heterologous promoter.

In one embodiment, the second polynucleotide is located downstream at the 3′ end of the first polynucleotide.

In another embodiment, the second polynucleotide is located upstream at the 5′ end of the first polynucleotide.

In one embodiment, the second polynucleotide is operably linked to a second promoter. The second promoter may be a homologous promoter, or a heterologous promoter. Preferably, the second promoter is a heterologous promoter.

In one embodiment, the first heterologous promoter comprises, or consists of a nucleic acid sequence having a sequence identity of at least 80%, e.g., at least 85%, at least 90%, at least 91%, at least 92%, at least 93%, at least 94%, at least 95%, at least 96%, at least 97%, at least 98%, at least 99%, or 100% to the nucleic acid sequence of the second promoter.

In another embodiment, the nucleic acid sequence of the first heterologous promoter and the nucleic acid sequence of the second promoter are identical.

In yet another embodiment, the first heterologous promoter comprises or consists of a nucleic acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 14.

In one embodiment, the second promoter comprises or consists of a nucleic acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 14.

In one embodiment the polypeptide of interest is secreted.

In another embodiment, the polypeptide of interest accumulated intracelluar, and is not being secreted. A non-limiting example for such a polypeptide of interest is an asparaginase.

In one embodiment, the host cell comprises at least two copies of the first heterologous promoter operably linked to the first polynucleotide, such as at least two copies, at least three copies, at least four copies, at least five copies, or at least six copies. Thereby the copy number of spoVG can be increased to an optimal level for improved polypeptide production. Alternative methods to fine-tune the expression of SpoVG include the use of a promoter library to identify promoters with suitable expression levels for SpoVG polypeptide expression.

In one embodiment, the SpoVG polypeptide, SpoVG fragment, or SpoVG variant is endogenous to the host cell.

In another embodiment, the SpoVG polypeptide, SpoVG fragment, or SpoVG variant is exogenous to the host cell. A non-limiting example is expression of a SpoVG polypeptide from B. subtilis in a B. licheniformis host cell.

In one embodiment, the SpoVG polypeptide, SpoVG fragment, or SpoVG variant comprises, or consists of an amino acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 2 or SEQ ID NO: 20.

In one embodiment, the first polynucleotide encoding the SpoVG polypeptide, SpoVG fragment, or SpoVG variant comprises, or consists of a nucleic acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 1, SEQ ID NO: 7, or SEQ ID NO: 19.

In another embodiment, the first polynucleotide encodes a stage 5 sporulation protein G (SpoVG) polypeptide, a SpoVG fragment or a SpoVG variant, AND a YabJ polypeptide, a YabJ fragment, or a YabJ variant. Non-limiting examples are disclosed by SEQ ID NO: 8 and SEQ ID NO: 9.

In a particular embodiment, the SpoVG polypeptide, SpoVG fragment, or SpoVG variant is translated into a polypeptide, fragment or variant not sharing the same polypeptide chain with the YabJ polypeptide, YabJ fragment, or YabJ variant. Non-limiting examples are disclosed by SEQ ID NO: 8 and SEQ ID NO: 9.

In one embodiment, the polynucleotide encoding the YabJ polypeptide, YabJ fragment, or YabJ variant is located upstream at the 5′ end of the polynucleotide encoding the SpoVG polypeptide, SpoVG fragment, or SpoVG variant. Non-limiting examples are disclosed by SEQ ID NO: 8 and SEQ ID NO: 9.

In another embodiment, the polynucleotide encoding the YabJ polypeptide, YabJ fragment, or YabJ variant is located downstream at the 3′ end of the polynucleotide encoding the SpoVG polypeptide, SpoVG fragment, or SpoVG variant.

In one embodiment, the YabJ polypeptide, YabJ fragment, or YabJ variant comprises, or consists of an amino acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 6. In one embodiment, the polynucleotide encoding the YabJ polypeptide, YabJ fragment, or YabJ variant comprises, or consists of a nucleic acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 5 or SEQ ID NO: 10. In one embodiment, the first polynucleotide comprises or consists of a nucleic acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 8 or SEQ ID NO: 9.

In a particular embodiment, the first polynucleotide and/or the second polynucleotide is/are operably linked in translational fusion with a third polynucleotide encoding a signal peptide. The third polynucleotide may be operably linked in translational fusion with the first and/or the second polypeptide. Preferably, the third polynucleotide encodes a signal peptide comprising, or consisting of an amino acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 16. In another embodiment, the host cell comprises in its genome a polynucleotide comprising from its 5′ end to its 3′ end:
a) the first heterologous promoter,
b) the first polynucleotide encoding the SpoVG polypeptide, and optionally the YabJ polypeptide, operably linked to the first heterologous promoter, and
c) the second polynucleotide encoding the polypeptide of interest. This enables easy integration into the genome and co-expression of both SpoVG and the polypeptide of interest, e.g. expression from the same cassette in a controlled ratio of spoVG gene copy number and copy number of the second polynucleotide.

In another embodiment, the host cell comprises in its genome a polynucleotide comprising from its 5′ end to its 3′ end:
a) the first heterologous promoter,
b) the second polynucleotide encoding the polypeptide of interest, operably linked to the first heterologous promoter, and
c) the first polynucleotide encoding the SpoVG polypeptide, and optionally the YabJ polypeptide. This enables easy integration into the genome and co-expression of both SpoVG and the polypeptide of interest, e.g. expression from the same cassette in a controlled ratio of spoVG gene copy number and copy number of the second polynucleotide.

Preferably, the polynucleotide encoding the elements a), b) and c) comprises, or consists of a polynucleotide having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 13 or SEQ ID NO: 12.

In another embodiment, the host cell comprises in its genome a polynucleotide comprising from its 5′ end to its 3′ end:
a) the first polynucleotide encoding the SpoVG polypeptide, and optionally the YabJ polypeptide, and
b) the second polynucleotide encoding the polypeptide of interest.

In another embodiment, the host cell comprises in its genome a polynucleotide comprising from its 5′ end to its 3′ end:
a) the second polynucleotide encoding the polypeptide of interest, and
b) the first polynucleotide encoding the SpoVG polypeptide, and optionally the YabJ polypeptide. Preferably, the polynucleotide encoding the elements a) and b) comprises, or consists of a polynucleotide having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 11 or SEQ ID NO: 12.

In another embodiment, the host cell comprises in its genome a polynucleotide comprising from its 5′ end to its 3′ end:
a) the first heterologous promoter, and
b) the first polynucleotide encoding the SpoVG polypeptide, and optionally the YabJ polypeptide, operably linked to the first heterologous promoter.

This embodiment enables genomic integration of the first polynucleotide separate from the genomic integration of the second polypeptide, thus the copy number of the first polynucleotide can be adjusted independent from the copy number of the second polynucleotide.

Preferably, the polynucleotide encoding the elements a) and b) comprises, or consists of a polynucleotide having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 21, SEQ ID NO:22, or SEQ ID NO: 23.

In one embodiment, the host cell comprises in its genome at least two copies of the second polynucleotide, such as at least two copies, at least three copies, at least four copies, at least five copies, or at least six copies. Increased copy number of the second polynucleotide can be used to optimize, i.e. increase, expression of the polypeptide of interest.

In one embodiment, the first heterologous promoter operably linked to the first polynucleotide is endogenous to the host cell. In another embodiment, the first heterologous promoter operably linked to the first polynucleotide is exogenous to the host cell. Additionally or alternatively, the promoter operably linked to the first polynucleotide is the native spoVG promoter.

In one embodiment, the host cell's total mRNA of the first polynucleotide encoding the SpoVG polypeptide, the SpoVG fragment, and/or the SpoVG variant is increased relative to total mRNA of the native SpoVG gene SpoVG polypeptide, the SpoVG fragment, and/or the SpoVG variant in a parent host cell which does not comprise the first polynucleotide operably linked to the first heterologous promoter, when cultivated under identical conditions. Preferably, the parent host cell is otherwise isogenic to the host cell of the first aspect.

In one embodiment, the host cell's total mRNA of the first polynucleotide encoding the SpoVG polypeptide, the SpoVG fragment, and/or the SpoVG variant is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, at least 215%, at least 220%, at least 225%, at least 230%, at least 235%, at least 240%, at least 245%, at least 250%, at least 255%, at least 260%, at least 265%, at least 270%, at least 275%, at least 280%, at least 285%, at least 290%, at least 295%, at least 300%, at least 305%, at least 310%, at least 315%, at least 320%, at least 325%, at least 330%, at least 335%, at least 340%, at least 345%, at least 350%, at least 355%, at least 360%, at least 365%, at least 370%, at least 375%, at least 380%, at least 385%, at least 390%, at least 395%, or at least 400%.

In one embodiment, the expression of the SpoVG polypeptide, the SpoVG fragment, and/or the SpoVG variant is increased relative to the expression of the native SpoVG polypeptide, the SpoVG fragment, and/or the SpoVG variant in a parent host cell which does not comprise the first polynucleotide operably linked to the first heterologous promoter, when cultivated under identical conditions. Preferably, the parent host cell is otherwise isogenic to the host cell according to the first aspect.

In one embodiment, the expression of the SpoVG polypeptide, the SpoVG fragment, and/or the SpoVG variant is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, at least 215%, at least 220%, at least 225%, at least 230%, at least 235%, at least 240%, at least 245%, at least 250%, at least 255%, at least 260%, at least 265%, at least 270%, at least 275%, at least 280%, at least 285%, at least 290%, at least 295%, at least 300%, at least 305%, at least 310%, at least 315%, at least 320%, at least 325%, at least 330%, at least 335%, at least 340%, at least 345%, at least 350%, at least 355%, at least 360%, at least 365%, at least 370%, at least 375%, at least 380%, at least 385%, at least 390%, at least 395%, or at least 400%.

In one embodiment, the expression of the polypeptide of interest is increased by at least 5%, at least 9%, at least 10%, at least 13%, at least 15%, at least 18%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, at least 215%, at least 220%, at least 225%, at least 230%, at least 235%, at least 240%, at least 245%, at least 250%, at least 255%, at least 260%, at least 265%, at least 270%, at least 275%, at least 280%, at least 285%, at least 290%, at least 295%, at least 300%, at least 305%, at least 310%, at least 315%, at least 320%, at least 325%, at least 330%, at least 335%, at least 340%, at least 345%, at least 350%, at least 355%, at least 360%, at least 365%, at least 370%, at least 375%, at least 380%, at least 385%, at least 390%, at least 395%, or at least 400%, relative to the expression of the polypeptide of interest in a parent host cell which does not comprise the first polynucleotide operably linked to the first heterologous promoter, when cultivated under identical conditions. Preferably, the parent host cell is otherwise isogenic to the host cell according to the first aspect.

In one embodiment the increased expression of the polypeptide of interest is achieved after 24 hours, 48 hours, 72 hours, 96 hours, or 120 hours of cultivation.

In a particular embodiment, the increased expression of the polypeptide of interest is achieved after 120 hours, or after at least 120 hours of cultivation.

In one embodiment, the host cell is a Gram-negative bacteria selected from the group consisting of Campylobacter, E. coli, Flavobacterium, Fusobacterium, Helicobacter, Ilyobacter, Neisseria, Pseudomonas, Salmonella, and Ureaplasma cells, or wherein the host cell is a Gram-positive cell selected from the group consisting of Bacillus, Clostridium, Enterococcus, Geobacillus, Lactobacillus, Lactococcus, Oceanobacillus, Staphylococcus, Streptococcus, or Streptomyces cells, such as Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus thuringiensis, Streptococcus equisimilis, Streptococcus pyogenes, Streptococcus uberis, and Streptococcus equi subsp. Zooepidemicus, Streptomyces achromogenes, Streptomyces avermitilis, Streptomyces coelicolor, Streptomyces griseus, and Streptomyces lividans cells, preferably the host cell is a Bacillus cell, most preferably a Bacillus subtilis or a Bacillus licheniformis cell.

In a particular embodiment, the host cell is a Bacillus cell.

In another embodiment, the host cell is a Bacillus subtilis cell.

In another embodiment, the host cell is a Bacillus licheniformis cell.

In one embodiment, the one or more polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deamidase, deoxyribonuclease, endoglucanase, esterase, alpha-galactosidase, beta-galactosidase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase; even more preferably the one or more polypeptide of interest comprises a cutinase.

In a preferred embodiment, the polypeptide of interest comprises a cutinase.

Preferably, the cutinase comprises, consists essentially of, or consists of the mature polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 4.

Preferably, the cutinase coding sequence comprises, consists essentially of, or consists of the polynucleotide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 3.

In another aspect, the present invention relates to a recombinant host cell comprising in its genome a nucleic acid construct according to the third aspect, and/or an expression vector according to the fourth aspect.

A construct or vector comprising a polynucleotide is introduced into a host cell so that the construct or vector is maintained as a chromosomal integrant or as a self-replicating extra-chromosomal vector as described earlier. The choice of a host cell will to a large extent depend upon the gene encoding the polypeptide and its source. The polypeptide can be native or heterologous to the recombinant host cell. Also, at least one of the one or more control sequences can be heterologous to the polynucleotide encoding the polypeptide.

The host cell may be any microbial cell useful in the recombinant production of a polypeptide of the present invention, e.g., a prokaryotic cell.

For purposes of this invention, Bacillus classes/genera/species shall be defined as described in Patel and Gupta, 2020, Int. J. Syst. Evol. Microbiol. 70: 406-438.

Methods for introducing DNA into prokaryotic host cells are well-known in the art, and any suitable method can be used including but not limited to protoplast transformation, competent cell transformation, electroporation, conjugation, transduction, with DNA introduced as linearized or as circular polynucleotide. Persons skilled in the art will be readily able to identify a suitable method for introducing DNA into a given prokaryotic cell depending, e.g., on the genus. Methods for introducing DNA into prokaryotic host cells are for example described in Heinze et al., 2018, BMC Microbiology 18:56, Burke et al., 2001, Proc. Natl. Acad. Sci. USA 98: 6289-6294, Choi et al., 2006, J. Microbiol. Methods 64: 391-397, and Donald et al., 2013, J. Bacteriol. 195(11): 2612-2620.

In an aspect, the host cell is isolated.

In another aspect, the host cell is purified.

### Methods of Production

In a second aspect, the invention relates to a method for producing one or more polypeptide of interest, the method comprising:
a) providing a bacterial host cell according to the first aspect,
b) cultivating said host cell under conditions conducive for expression of the one or more polypeptide of interest; and,
c) optionally recovering the one or more polypeptide of interest.

The method may be performed batch-wise or continuously. While it is principally possible that a subsequent step commences before the preceding step has been terminated, the individual steps a), b) and c) are preferably performed consecutively in alphabetical order, wherein a subsequent step commences after the preceding step has been completely terminated. It is also contemplated, however, that additional intermediate steps which are not mentioned among steps a), b) and c) are performed in between any of steps a), b), and/or c).

In one aspect, the cell is a Bacillus cell. In another aspect, the cell is a Bacillus licheniformis cell. In another aspect, the cell is a Bacillus licheniformis cell, ATCC14580.

The host cell is cultivated in a nutrient medium suitable for production of the polypeptide using methods known in the art. For example, the cell may be cultivated by shake flask cultivation, or small-scale or large-scale fermentation (including continuous, batch, fed-batch, or solid-state, and/or microcarrier-based fermentations) in laboratory or industrial fermentors in a suitable medium and under conditions allowing the polypeptide to be expressed and/or isolated. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

In one embodiment, the cultivation is a fed-batch process.

In one embodiment, the cultivation is carried out over a period of at least 48 hours, at least 72 hours, at least 96 hours, or at least 120 hours.

The polypeptide may be detected using methods known in the art that are specific for the polypeptide, including, but not limited to, the use of specific antibodies, formation of an enzyme product, disappearance of an enzyme substrate, or an assay determining the relative or specific activity of the polypeptide.

The polypeptide may be recovered from the medium using methods known in the art, including, but not limited to, collection, centrifugation, filtration, extraction, spray-drying, evaporation, or precipitation. In one aspect, a whole fermentation broth comprising the polypeptide is recovered. In another aspect, a cell-free fermentation broth comprising the polypeptide is recovered.

The polypeptide may be purified by a variety of procedures known in the art to obtain substantially pure polypeptides and/or polypeptide fragments (see, e.g., Wingfield, 2015, Current Protocols in Protein Science; 80(1): 6.1.1-6.1.35; Labrou, 2014, Protein Downstream Processing, 1129: 3-10).

In an alternative aspect, the polypeptide is not recovered.

### Polynucleotides

The present invention also relates to polynucleotides encoding a polypeptide of the present invention, as described herein.

The polynucleotide may be a genomic DNA, a cDNA, a synthetic DNA, a synthetic RNA, a mRNA, or a combination thereof. The polynucleotide may be cloned from a strain of Bacillus, or a related organism and thus, for example, may be a polynucleotide sequence encoding a variant of the polypeptide of the invention.

In an embodiment, the polynucleotide is a subsequence encoding a fragment having SpoVG activity and/or DNA-, RNA-, and/or protein-binding activity of the present invention. In an aspect, the subsequence contains at least 66 nucleotides (e.g., nucleotides 148 to 213 of SEQ ID NO: 1), at least 72 nucleotides (e.g., nucleotides 145 to 216 of SEQ ID NO: 1), or at least 78 nucleotides (e.g., nucleotides 140 to 219 of SEQ ID NO: 1).

In one embodiment the polynucleotide encoding the polypeptide of the present invention is isolated from a Bacillus cell.

The polynucleotide may also be mutated by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

In an aspect, the polynucleotide is isolated.

In another aspect, the polynucleotide is purified.

### Nucleic Acid Constructs

The present invention also relates to nucleic acid constructs comprising a polynucleotide of the present invention, wherein the polynucleotide is operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. Techniques for modifying polynucleotides utilizing recombinant DNA methods are well known in the art.

In a third aspect, the invention relates to a nucleic acid construct comprising at least one first heterologous promoter operably linked to at least one first polynucleotide encoding a stage sporulation protein G (SpoVG) polypeptide, a SpoVG fragment, or a SpoVG variant.

In one embodiment the SpoVG polypeptide is a SpoVG fragment or a SpoVG variant.

In one embodiment, the nucleic acid construct is further comprising at least one second polynucleotide encoding at least one polypeptide of interest. This enables co-expression of SpoVG together with the polypeptide of interest.

In one embodiment, the second polynucleotide is operably linked to the first heterologous promoter. Thereby SpoVG and the polypeptide of interest are co-expressed and regulated under the same promoter, i.e. comprised in one expression cassette.

In another embodiment the second polynucleotide is located downstream at the 3′ end of the first polynucleotide.

In yet another embodiment, the second polynucleotide is located upstream at the 5′ end of the first polynucleotide.

In one embodiment the second polynucleotide is operably linked to a second promoter, preferably the second promoter is a heterologous promoter. Thereby the polypeptide of interest can be expressed under the control of a promoter which is only regulating the expression of the polypeptide of interest. This also enables to express the polypeptide of interest and the SpoVG from different expression cassettes.

In one embodiment, the first heterologous promoter comprises, or consists of a nucleic acid sequence having a sequence identity of at least 80%, e.g., at least 85%, at least 90%, at least 91%, at least 92%, at least 93%, at least 94%, at least 95%, at least 96%, at least 97%, at least 98%, at least 99%, or 100% to the nucleic acid sequence of the second promoter.

In one embodiment, the nucleic acid sequence of the first heterologous promoter and the nucleic acid sequence of the second promoter are identical.

In one embodiment, the first heterologous promoter comprises or consists of a nucleic acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 14.

In one embodiment, the second promoter comprises or consists of a nucleic acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 14.

In one embodiment, the SpoVG polypeptide, SpoVG fragment, or SpoVG variant comprises, or consists of an amino acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 2 or SEQ ID NO: 20.

In one embodiment, the first polynucleotide encoding the SpoVG polypeptide, SpoVG fragment, or SpoVG variant comprises, or consists of a nucleic acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 1, SEQ ID NO: 7, or SEQ ID NO: 19.

In another embodiment, the first polynucleotide encodes a stage 5 sporulation protein G (SpoVG) polypeptide, a SpoVG fragment or a SpoVG variant, AND a YabJ polypeptide, a YabJ fragment, or a YabJ variant. A non-limiting example is depicted by SEQ ID NO: 8 and SEQ ID NO: 9.

In yet another embodiment, the SpoVG polypeptide, SpoVG fragment, or SpoVG variant is translated into a polypeptide, fragment or variant not sharing the same polypeptide chain with the YabJ polypeptide, YabJ fragment, orYabJ variant. A non-limiting example is depicted by SEQ ID NO: 8 and SEQ ID NO: 9.

In one embodiment, the polynucleotide encoding the YabJ polypeptide, YabJ fragment, or YabJ variant is located upstream at the 5′ end of the polynucleotide encoding the SpoVG polypeptide, SpoVG fragment, or SpoVG variant. A non-limiting example is depicted by SEQ ID NO: 8 and SEQ ID NO: 9.

In another embodiment, T the polynucleotide encoding the YabJ polypeptide, YabJ fragment, or YabJ variant is located downstream at the 3′ end of the polynucleotide encoding the SpoVG polypeptide, SpoVG fragment, or SpoVG variant.

In one embodiment, the YabJ polypeptide, YabJ fragment, or YabJ variant comprises, or consists of an amino acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 6.

In one embodiment, the polynucleotide encoding the YabJ polypeptide, YabJ fragment, or YabJ variant comprises, or consists of a nucleic acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 5 or SEQ ID NO: 10.

In another embodiment, the first polynucleotide comprises or consists of a nucleic acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 8 or SEQ ID NO: 9.

In one embodiment, the nucleic acid construct comprises a polynucleotide comprising from its 5′ end to its 3′ end:
a) the first heterologous promoter,
b) the first polynucleotide encoding the SpoVG polypeptide, and optionally the YabJ polypeptide, operably linked to the first heterologous promoter, and
c) the second polynucleotide encoding the polypeptide of interest.

In another embodiment, the nucleic acid construct comprises a polynucleotide comprising from its 5′ end to its 3′ end:
a) the first heterologous promoter,
b) the second polynucleotide encoding the polypeptide of interest, operably linked to the first heterologous promoter, and
c) the first polynucleotide encoding the SpoVG polypeptide, and optionally the YabJ polypeptide. Preferably, the polynucleotide encoding the elements a), b) and c) comprises, or consists of a polynucleotide having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 13 or SEQ ID NO: 12.

In yet another embodiment, the nucleic acid construct comprises a polynucleotide comprising from its 5′ end to its 3′ end:
a) the first polynucleotide encoding the SpoVG polypeptide, and optionally the YabJ polypeptide, and
b) the second polynucleotide encoding the polypeptide of interest.

In another embodiment, the nucleic acid construct comprises a polynucleotide comprising from its 5′ end to its 3′ end:
a) the second polynucleotide encoding the polypeptide of interest, and
b) the first polynucleotide encoding the SpoVG polypeptide, and optionally the YabJ polypeptide. Preferably, the polynucleotide encoding the elements a) and b) comprises, or consists of a polynucleotide having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 11 or SEQ ID NO: 12.

In another embodiment, the nucleic acid construct comprises a polynucleotide comprising from its 5′ end to its 3′ end:
a) the first heterologous promoter, and
b) the first polynucleotide encoding the SpoVG polypeptide, and optionally the YabJ polypeptide, operably linked to the first heterologous promoter. Preferably, the polynucleotide encoding the elements a) and b) comprises, or consists of a polynucleotide having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 21, SEQ ID NO: 22, or SEQ ID NO: 23.

In one embodiment, the one or more polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deamidase, deoxyribonuclease, endoglucanase, esterase, alpha-galactosidase, beta-galactosidase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase; even more preferably the one or more polypeptide of interest comprises a cutinase.

In another embodiment, the polypeptide of interest comprises a cutinase.

In a particular embodiment, the one or more polypeptide of interest is a cutinase, such as a cutinase which comprises, consists essentially of, or consists of the mature polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 4.

### Promoters

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are described in Sambrook et al., 1989, Molecular Cloning: A Laboratory Manual, Cold Spring Harbor Lab., NY, Davis et al., 2012, supra, and Song et al., 2016, PLOS One 11(7): e0158447.

In one embodiment the promoter comprises or consists of a polynucleotide having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 14.

### Terminators

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells may be obtained from the genes for Bacillus clausii alkaline protease (aprH), Bacillus licheniformis alpha-amylase (amyL), and Escherichia coli ribosomal RNA (rrnB).

In one embodiment, the terminator is comprising or consisting of a polynucleotide having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 17.

mRNA Stabilizers

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cry/I/A gene (WO 94/25612) and a Bacillus subtilis SP82 gene (Hue et al., 1995, J. Bacteriol. 177: 3465-3471).

Examples of mRNA stabilizer regions for fungal cells are described in Geisberg et al., 2014, Cell 156(4): 812-824, and in Morozov et al., 2006, Eukaryotic Cell 5(11): 1838-1846.

In one embodiment, the mRNA stabilizer is comprising or consisting of a polynucleotide having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 18.

### Leader Sequences

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

Suitable leaders for bacterial host cells are described by Hambraeus et al., 2000, Microbiology 146(12): 3051-3059, and by Kaberdin and Blssi, 2006, FEMS Microbiol. Rev. 30(6): 967-979.

### Polyadenylation Sequences

The control sequence may also be a polyadenylation sequence, a sequence operably linked to the 3′-terminus of the polynucleotide which, when transcribed, is recognized by the host cell as a signal to add polyadenosine residues to transcribed mRNA. Any polyadenylation sequence that is functional in the host cell may be used.

### Signal Peptides

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the polypeptide. Any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Freudl, 2018, Microbial Cell Factories 17: 52.

In a particular embodiment, the first polynucleotide and/or the second polynucleotide is/are operably linked in translational fusion with a third polynucleotide encoding a signal peptide.

In one embodiment, the third polynucleotide is operably linked in translational fusion with the first and/or the second polypeptide.

In one embodiment, the third polynucleotide encoding the signal peptide is comprising, or consisting of a polynucleotide having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 15.

In one embodiment, the third polynucleotide encodes a signal peptide comprising, or consisting of an amino acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 16.

### Propeptides

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence. Additionally or alternatively, when both signal peptide and propeptide sequences are present, the polypeptide may comprise only a part of the signal peptide sequence and/or only a part of the propeptide sequence. Alternatively, the final or isolated polypeptide may comprise a mixture of mature polypeptides and polypeptides which comprise, either partly or in full length, a propeptide sequence and/or a signal peptide sequence.

### Regulatory Sequences

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. Other examples of regulatory sequences are those that allow for gene amplification.

### Transcription Factors

The control sequence may also be a transcription factor, a polynucleotide encoding a polynucleotide-specific DNA-binding polypeptide that controls the rate of the transcription of genetic information from DNA to mRNA by binding to a specific polynucleotide sequence. The transcription factor may function alone and/or together with one or more other polypeptides or transcription factors in a complex by promoting or blocking the recruitment of RNA polymerase. Transcription factors are characterized by comprising at least one DNA-binding domain which often attaches to a specific DNA sequence adjacent to the genetic elements which are regulated by the transcription factor. The transcription factor may regulate the expression of a protein of interest either directly, i.e., by activating the transcription of the gene encoding the protein of interest by binding to its promoter, or indirectly, i.e., by activating the transcription of a further transcription factor which regulates the transcription of the gene encoding the protein of interest, such as by binding to the promoter of the further transcription factor. Suitable transcription factors for fungal host cells are described in WO 2017/144177. Suitable transcription factors for prokaryotic host cells are described in Seshasayee et al., 2011, Subcellular Biochemistry 52: 7-23, as well in Balleza et al., 2009, FEMS Microbiol. Rev. 33(1): 133-151.

### Expression Vectors

In a fourth aspect, the present invention relates to an expression vector comprising a nucleic acid construct according to the third aspect.

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

The vector preferably contains at least one element that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous recombination, such as homology-directed repair (HDR), or non-homologous recombination, such as non-homologous end-joining (NHEJ).

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

More than one copy of a polynucleotide of the present invention may be inserted into a host cell to increase production of a polypeptide. For example, 2 or 3 or 4 or 5 or more copies are inserted into a host cell. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

### Methods of Improving Protein Expression in a Host Cell

In a fifth aspect, the invention relates to a method for production of a recombinant bacterial host cell with increased yield of a polypeptide of interest, the method comprising:
a) providing a parent bacterial host cell comprising in its genome a native polynucleotide encoding a SpoVG polypeptide, SpoVG variant, or SpoVG fragment,
b) introducing into said parent bacterial host cell a first polynucleotide encoding the SpoVG polypeptide, fragment or variant, and
c) introducing into said parent bacterial host cell a second polynucleotide encoding the polypeptide of interest,
wherein step c) is carried out prior to step b), in parallel to step b), or after step b), and wherein the recombinant bacterial host cell comprises increased expression of the SpoVG polypeptide, fragment or variant relative to the parent bacterial host cell when cultivated under identical conditions. Preferably, a polynucleotide construct according to the third aspect, and/or an expression vector according to the fourth aspect is introduced into the host cell during step b) and/or step c).

The method may be performed batch-wise or continuously. While it is principally possible that a subsequent step commences before the preceding step has been terminated, the individual steps a), b) and c) are preferably performed consecutively in alphabetical order, wherein a subsequent step commences after the preceding step has been completely terminated. It is also contemplated, however, that additional intermediate steps which are not mentioned among steps a), b) and c) are performed in between any of steps a), b), and/or c).

In another aspect, the invention relates to a method for production of a recombinant bacterial host cell with increased yield of a polypeptide of interest, the method comprising:
a) providing a parent bacterial host cell comprising in its genome a native polynucleotide encoding a SpoVG polypeptide, SpoVG variant, or SpoVG fragment, and a second polynucleotide encoding a polypeptide of interest, and
b) introducing into said parent bacterial host cell a first polynucleotide encoding the SpoVG polypeptide, fragment or variant, and
wherein the recombinant bacterial host cell comprises increased expression of the SpoVG polypeptide, fragment or variant relative to the parent bacterial host cell when cultivated under identical conditions. Preferably, a polynucleotide construct according to the third aspect, and/or an expression vector according to the fourth aspect is introduced into the host cell during step b). The method may be performed batch-wise or continuously. While it is principally possible that a subsequent step commences before the preceding step has been terminated, the individual steps a) and b) are preferably performed consecutively in alphabetical order, wherein a subsequent step commences after the preceding step has been completely terminated. It is also contemplated, however, that additional intermediate steps which are not mentioned among steps a) and b) are performed in between any of steps a), and b).

In one embodiment, the SpoVG polypeptide, SpoVG fragment, or SpoVG variant comprises, or consists of an amino acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 2 or SEQ ID NO: 20.

In one embodiment, the first polynucleotide encoding the SpoVG polypeptide, SpoVG fragment, or SpoVG variant comprises, or consists of a nucleic acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 1, SEQ ID NO: 7, or SEQ ID NO: 19.

In one embodiment, step b) results in a recombinant host cell comprising at least two copies of the first heterologous promoter operably linked to the first polynucleotide, such as at least two copies, at least three copies, at least four copies, at least five copies, or at least six copies.

In one embodiment, the expression of the polypeptide of interest is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, at least 215%, at least 220%, at least 225%, at least 230%, at least 235%, at least 240%, at least 245%, at least 250%, at least 255%, at least 260%, at least 265%, at least 270%, at least 275%, at least 280%, at least 285%, at least 290%, at least 295%, at least 300%, at least 305%, at least 310%, at least 315%, at least 320%, at least 325%, at least 330%, at least 335%, at least 340%, at least 345%, at least 350%, at least 355%, at least 360%, at least 365%, at least 370%, at least 375%, at least 380%, at least 385%, at least 390%, at least 395%, or at least 400%, relative to the expression of the polypeptide of interest in a parent host cell which does not comprise the first polynucleotide operably linked to the first heterologous promoter, when cultivated under identical conditions. Preferably, the parent host cell is otherwise isogenic to the host cell according to the first aspect.

In one embodiment the increased expression of the polypeptide of interest is achieved after 24 hours, 48 hours, 72 hours, 96 hours, or 120 hours of cultivation.

In one embodiment, the increased expression of the polypeptide of interest is achieved after 120 hours, or after at least 120 hours of cultivation.

In one embodiment the increased expression/yield is achieved in a fed-batch cultivation.

In one embodiment, the host cell is a Gram-negative bacteria selected from the group consisting of Campylobacter, E. coli, Flavobacterium, Fusobacterium, Helicobacter, Ilyobacter, Neisseria, Pseudomonas, Salmonella, and Ureaplasma cells, or wherein the host cell is a Gram-positive cell selected from the group consisting of Bacillus, Clostridium, Enterococcus, Geobacillus, Lactobacillus, Lactococcus, Oceanobacillus, Staphylococcus, Streptococcus, or Streptomyces cells, such as Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus thuringiensis, Streptococcus equisimilis, Streptococcus pyogenes, Streptococcus uberis, and Streptococcus equi subsp. Zooepidemicus, Streptomyces achromogenes, Streptomyces avermitilis, Streptomyces coelicolor, Streptomyces griseus, and Streptomyces lividans cells, preferably the host cell is a Bacillus cell, most preferably a Bacillus subtilis or a Bacillus licheniformis cell.

In a particular embodiment, the host cell is a Bacillus cell.

In another embodiment, the host cell is a Bacillus subtilis cell.

In another embodiment, the host cell is a Bacillus licheniformis cell.

In one embodiment, the one or more polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deamidase, deoxyribonuclease, endoglucanase, esterase, alpha-galactosidase, beta-galactosidase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase; even more preferably the one or more polypeptide of interest comprises a cutinase.

In a preferred embodiment, the polypeptide of interest comprises a cutinase.

Preferably, the cutinase comprises, consists essentially of, or consists of the mature polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 4.

### Polypeptides Having SpoVG Activity

The present invention relates to polypeptides having SpoVG activity. In an aspect, the invention relates to polypeptides having SpoVG activity, selected from the group consisting of:
(a) a polypeptide having at least 60% sequence identity to SEQ ID NO: 2 or SEQ ID NO: 20;
(b) a polypeptide encoded by a polynucleotide having at least 60% sequence identity to the mature polypeptide coding sequence of SEQ ID NO: 1 or SEQ ID NO: 19;
(c) a polypeptide derived from SEQ ID NO: 2 or SEQ ID NO: 20, or a mature polypeptide of SEQ ID NO: 2 or SEQ ID NO: 20 by substitution, deletion or addition of one or several amino acids;
(d) a polypeptide derived from the polypeptide of (a), (b), or (c) wherein the N- and/or C-terminal end has been extended by the addition of one or more amino acids; and
(e) a fragment of the polypeptide of (a), (b), (c), or (d);
   wherein the polypeptide has SpoVG activity.

In an aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 2 or SEQ ID NO: 20, or a mature polypeptide of SEQ ID NO: 2 or SEQ ID NO: 20.

The polypeptide preferably comprises, consists essentially of, or consists of the amino acid sequence of SEQ ID NO: 2 or SEQ ID NO: 20, or a mature polypeptide thereof.

The polypeptide may have an N-terminal and/or C-terminal extension of one or more amino acids, e.g., 1-5 amino acids.

In another aspect, the polypeptide is a fragment containing at least 22 amino acid residues (e.g., amino acids 50 to 71 of SEQ ID NO: 2, “KRTPDGEFRDIAHPINSSTRGK”), at least 24 amino acid residues (e.g., amino acids 49 to 72 of SEQ ID NO: 2, “SKRTPDGEFRDIAHPINSSTRGKI”), or at least 26 amino acid residues (e.g., amino acids 48 to 73 of SEQ ID NO: 2, “PSKRTPDGEFRDIAHPINSSTRGKIQ”). Such fragment comprises the amino acids involved in SpoVG-DNA interaction and the amino acids involved in DNA sequence specificity.

In some embodiments, the polypeptide is encoded by a polynucleotide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 1 or SEQ ID NO: 19.

The polynucleotide encoding the polypeptide preferably comprises, consists essentially of, or consists of nucleotides 1 to 291 of SEQ ID NO: 1 or SEQ ID NO: 19.

In another aspect, the polypeptide is derived from SEQ ID NO: 2 or SEQ ID NO: 20 by substitution, deletion or addition of one or several amino acids. In another aspect, the polypeptide is derived from a mature polypeptide of SEQ ID NO: 2 or SEQ ID NO: 20 by substitution, deletion or addition of one or several amino acids. In some embodiments, the polypeptide is a variant of SEQ ID NO: 2 or SEQ ID NO: 20 comprising a substitution, deletion, and/or insertion at one or more positions. In one aspect, the number of amino acid substitutions, deletions and/or insertions introduced into the polypeptide of SEQ ID NO: 2 or SEQ ID NO: 20 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for SpoVG activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596: 583-589.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a fusion polypeptide.

In an aspect, the polypeptide is isolated.

In another aspect, the polypeptide is purified.

### Sources of Polypeptides Having SpoVG Activity

A polypeptide having SpoVG activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide of the invention has been inserted. In one aspect, the polypeptide obtained from a given source is secreted extracellularly.

In another aspect, the polypeptide is a polypeptide obtained from a Bacillus, such as a Bacillus licheniformis, e.g., a polypeptide obtained from Bacillus licheniformis, ATCC14580.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

The polypeptides may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Davis et al., 2012, Basic Methods in Molecular Biology, Elsevier).

The present invention is further described by the following examples that should not be construed as limiting the scope of the invention.

### EXAMPLES

### Strains

AEB1517: This strain is a B. subtilis donor strain for conjugation of B. licheniformis as described previously (see U.S. Pat. Nos. 5,695,976, 5,733,753, 5,843,720, 5,882,888, and WO2006042548). The strain contains pLS20 and the methylase gene M.blil 904II (US20130177942) expressed from a triple promoter (SEQ ID NO: 14) at the amyE locus, the pBC16-derived or beta and the B. subtilis comS gene (and a kanamycin resistance gene) are expressed from a triple promoter at the air locus (making the strain D-alanine requiring).
PP3724: AEB1517 derivative where a second gene cassette consisting of the comS gene expressed from a triple promoter (SEQ ID NO: 14) is inserted at the pel locus (pectate lyase) (see US20190276855).
BT18049: PP3724 transformed with the plasmid pCLK015.
AN1302: A derivative of B. licheniformis Ca63 with seven deletions in the protease genes, aprL, mprL, bprAB, epr, wprA, vpr, ispA. Furthermore, a deletion in the spo gene spollAC, the cypX, sacB and forD. This strain harbors two P3-marker gene expression cassettes.
AN1301: A derivative of B. licheniformis Ca63 with seven deletions in the protease genes, aprL, mprL, bprAB, epr, wprA, vpr, ispA. Furthermore, a deletion in the spo gene spollAC, the cypX, sacB and forD. This strain harbors three P3-marker gene expression cassettes.
AEB1954: A derivative of B. licheniformis Ca63 with seven deletions in the protease genes, aprL, mprL, bprAB, epr, wprA, vpr, ispA. Furthermore, a deletion in the spo gene spollAC, the cypX, sacB and forD. This strain harbors five P3-marker gene expression cassettes.
BT18062: AN1302 with two copies of the expression cassette encoding SP32 signal peptide (SEQ ID NO: 16) fused to the gene encoding cutinase X1 (SEQ ID NO: 4). The two copies are inserted at the lacA2 locus and the xylA locus on the chromosome.
BKQ1867: A derivative of B. licheniformis Ca63 with seven deletions in the protease genes, aprL, mprL, bprAB, epr, wprA, vpr, ispA. Furthermore, a deletion in the spo gene spollAC, the cypX, sacB and forD. This strain harbors four P3-marker gene expression cassettes.
BT18062: AN1302 with two copies of the expression cassette encoding SP32 signal peptide (SEQ ID NO: 16) fused to the gene encoding cutinase X1 (SEQ ID NO: 4). The two copies are inserted at the lacA2 locus and the xylA locus on the chromosome.
BT18076: AEB1954 with three copies of the expression cassette encoding SP32 signal peptide fused to the cutinase X1. The three copies are inserted in the amyE locus, the bglC locus and the gntP locus on the chromosome.
BT18074: AEB1954 with four copies of the expression cassette encoding SP32 signal peptide fused to the cutinase X1. The four copies are inserted in the amyE locus, the bglC locus, the gntP locus and the xylA locus on the chromosome.
BT18068: AEB1954 with five copies of the expression cassette encoding SP32 signal peptide fused to the cutinase X1. The five copies are inserted in the amyE locus, the bglC locus, the gntP locus, the xylA locus and the lacA2 locus on the chromosome.
BT14205: AEB1954 with five copies of the expression cassette encoding SP32 signal peptide fused to the cutinase X1 and one extra copy of yabJ-spoVG. Cutinase copies are inserted in the amyE locus, the bglC locus, the gntP locus, the lacA2 locus and the Cutinase-yabJ-spoVG cassette in the xylA locus on the chromosome.
BT18093: AN1302 with 2 copies of the expression cassette encoding SP32 signal peptide fused to the cutinase variant X2. The two copies are inserted in the xylA locus and the lacA2 locus on the chromosome.
BT14271: AN1302 with 1 copy of the expression cassette encoding SP32 signal peptide fused to the cutinase variant X2 in the xylA locus and one copy of the expression cassette encoding SP32_cutinase X2_yabJ_spoVG in the lacA2 locus on the chromosome.
BT14265: AN1301 with 3 copies of the expression cassette encoding SP32 signal peptide fused to the cutinase variant X2 and one extra copy of yabJ-spoVG. Cutinase copies are inserted in the lacA2 and xylA loci, and the Cutinase-yabJ-spoVG in the bglC locus on the chromosome.
BT18064-1: AEB1954 with four copies of the expression cassette encoding SP32 signal peptide fused to the cutinase X1. The four copies are inserted in the amyE locus, the bglC locus, the gntP locus and the lacA2 locus on the chromosome. Also carries a P3-marker gene expression cassette in the xylA locus.
BT18064-2: AEB1954 with three copies of the expression cassette encoding SP32 signal peptide fused to the cutinase X1. The three copies are inserted in the amyE locus, the bglC locus, and the gntP locus on the chromosome. Also carries P3-marker gene expression cassettes in xylA and lacA2 loci.
PP3713: AEB1517+plasmid pPP3708
BT14199: PP3724+plasmid pBT14199
BT14316: PP3724+plasmid pBT14316
BT14317: PP3724+plasmid pBT14317
BT14255: PP3724+plasmid pBT14255
BT14285: PP3724+plasmid pBT14289
BT18089: PP3724+plasmid pBT18089

### Plasmids

pAEB267: A pE194 derivative plasmid with a minimal attP site of TP901-1, previously described in US20080085535.
pPP3708: pAEB267 derivative with attB used for removal of P3-marker gene expression cassettes from the chromosome.
pCLK015: pAEB267 derivative where the gene encoding the SP32 signal peptide is fused to the cutinase X1 gene.
pBT14199: pAEB267 derivative with a gene encoding cutinase in operon fusion with Bacillus licheniformis yabJ-spoVG.
pBT14285: pAEB267 derivative with a genes encoding Bacillus licheniformis yabJ and spoVG in operon fusion.
pBT14255: pAEB267 derivative with a gene encoding cutinase variant X2 in operon fusion with Bacillus licheniformis yabJ-spoVG.
pBT14316: pAEB267 derivative with a gene encoding Bacillus licheniformis spoVG.
pBT14317: pAEB267 derivative with a gene encoding Bacillus licheniformis yabJ.

TABLE 1
Sequence listing overview (AA = amino acid)

| SEQ | |
|---|---|
| ID NO: | description |
| 1 | coding sequence spoVG B. licheniformis |
| 2 | AA sequence SpoVG B. licheniformis |
| 3 | coding sequence cutinase |
| 4 | AA sequence cutinase |
| 5 | coding sequence for yabJ |
| 6 | AA sequence yabJ |
| 7 | coding sequence for BLP000052-spoVG |
| 8 | coding sequence yabJ-spoVG |
| 9 | coding sequence for yabJ-BLP00052-spoVG |
| 10 | coding sequence yabJ-BLP00052 |
| 11 | coding sequence for SP32-cutinase cassette − yabJ-BLP00052- |
| | spoVG − TamyL-attP |
| 12 | coding sequence for cutinase cassette + yabJ-BLP00052- |
| | spoVG − TamyL-attP |
| 13 | coding sequence for triple promoter-SP32-cutinase cassette + |
| | yabJ-BLP00052-spoVG − TamyL-attP |
| 14 | triple-promoter sequence |
| 15 | SP 32 coding sequence |
| 16 | SP32 amino acid sequence |
| 17 | TamyL coding sequence |
| 18 | cryllIA mRNA stabilizer region |
| 19 | SpoVG coding sequence B. subtilis |
| 20 | SpoVG AA sequence B. subtilis |
| 21 | coding sequence for triple promoter-yabJ-spoVG |
| 22 | coding sequence for triple promoter-spoVG-yabJ |
| 23 | coding sequence for triple promoter-spoVG |

### Media and Solutions Media:

LB agar: 10 g/l peptone from casein; 5 g/l yeast extract, 10 g/l sodium chloride; 12 g/l Bacto-agar adjusted to pH 7.0+/−0.2. Premix from Merck was used (LB-agar (Miller) 110283)
M-9 buffer: di-sodiumhydrogenphosphate, 2H₂O 8.8 g/l; potassiumdihydrogenphosphate 3 g/l; sodium chloride 4 g/l; magnesium sulphate, 7H₂O 0.2 g/l (deionized water is used in this buffer)
PRK-50: 110 g/l soy grits; di-sodiumhydrogenphosphate, 2H₂O 5 g/l; Antifoam (Struktol SB2121; Schill & Seilacher, Hamburg, Germany) 1 ml/l, pH adjusted to 8.0 with NaOH/H₂PO₄ before sterilization.
Make-up medium: Tryptone (Casein hydrolystae from Difco (Bacto™ Tryptone pancreatic Digest of Casein 211699) 30 g/l; magnesium sulphate, 7H₂O 4 g/l; di-potassiumdihydrogenphosphate 7 g/l; di-sodiumhydrogenphosphate, 2H₂O 7 g/l; di-ammoniumsulphate 4 g/l; citric acid 0.78 g/l; vitamins (thiamin-dichlorid 34.2 mg/l; riboflavin 2.9 mg/l; nicotinic acid 23 mg/l; calcium D-pantothenate 28.5 mg/l; pyridoxai-HCl 5.7 mg/l; D-biotin 1.1 mg/l; folic acid 2.9 mg/l); trace metals (MnSO₄, H₂O 39.2 mg/l, FeSO₄, 7H₂O 157 mg/l, CuSO₄, 5H₂O 15.6 mg/l; ZnCl₂ 15.6 mg/l); Antifoam (Struktol SB2121; Schill & Seilacher, Hamburg, Germany) 1.25 ml/l; pH adjusted to 6.0 with NaOH/H₂PO₄ before sterilization.
Feed-medium: Glucose, 1H₂O 820 g/l

### Procedure for Inoculum Steps

a) Grow the strain on LB agar slants o/n at 37° C.
b) Wash the agar with M-9 buffer and collect the cell suspension. Measure OD650.
c) Inoculate PRK-50 shake flask (OD650×ml cell suspension=1).
d) Incubate the shake flask at 300 rpm o/n at 37° C.
e) Start the main fermentor by adding the growing shake flask culture (10% of make-up media, i.e. 80 ml to 800 ml).

### Fermentor Equipment

Standard lab fermenters equipped with a temperature control system, pH control with ammoniawater and phosphoric acid, dissolved oxygen electrode to measure >20% oxygen saturation through the entire fermentation.

### Fermentation Parameters

Temperature: 37° C.
Keep pH between 6.8 and 7.2 using ammoniawater and phosphoric acid.
Aeration: 1.5 L/min/kg broth weight
Agitation: 1500 rpm.

### Cutinase Activity Assay

The cultivation samples from microplate fermentation or fed-batch fermentation were diluted. 20 μL of the diluted cultivation sample was transferred in technical duplicates to 96-well plates. A calibration curve with increasing concentrations of purified cutinase standard (SEQ ID NO:4, kindly provided by Carbios, France) was added to each 96 well plate. 180 ul of p-nitrophenyl palmitate (Sigma-Aldrich, Denmark) was added to the plate and the colorimetric reaction was measured in a Cytation5 plate reader at 405 nm, 23° C. for 5 min, measuring absorbance every 30 seconds.

### Example 1. Construction of Bacillus licheniformis Strain BT18062 Expressing Cutinase

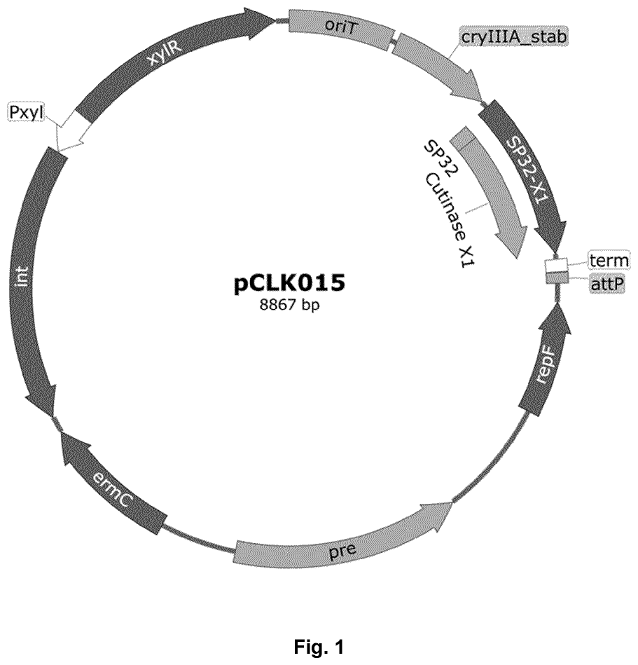

Plasmid pCLK015 was constructed for insertion of a gene encoding cutinase X1 with the signal peptide of Bacillus pumilus putative DUF3298 (designated by name SP32-X1) into the genome of a Bacillus subtilis host using the site-specific recombinase-mediated method described in WO 2018/077796. A map of pCLK015 is shown in FIG. 1, the DNA sequence encoding cutinase X1 is shown as SEQ ID NO: 3 and the polynucleotide sequence encoding the SP32 signal peptide is shown in SEQ ID NO: 15, and the corresponding amino acid sequence is shown in SEQ ID NO: 4 and SEQ ID NO: 16, respectively. Plasmid pCLK015 was introduced into conjugation donor strain Bacillus subtilis PP3724 by transformation, resulting in strain BT18049 (PP3724/pCLK015). Using conjugation donor strain BT18049, plasmid pCLK015 was introduced by conjugation into a derivative of Bacillus licheniformis AN1302 comprising two chromosomal target sites for insertion of the plasmid and deletions in the genes encoding alkaline protease (aprL), Glu-specific protease (mprL), bacillopeptidase F (bprAB), minor extracellular serine proteases (epr and vpr), secreted quality control protease (wprA) and intracellular serine protease (ispA). At each of the two chromosomal target sites of the B. licheniformis host is an expression cassette comprising a P3 promoter (SEQ ID NO: 14) followed by the crylllA mRNA stabilizer region (SEQ ID NO:18), a fluorescent marker gene and an attB recombination site. The plasmid inserted into the B. licheniformis chromosome by site-specific recombination between the attP sites on the plasmid, and attB sites at the target chromosomal loci. The plasmid was then allowed to excise from the chromosome via homologous recombination by incubation at 34° C. in the absence of erythromycin selection. Integrants that had lost the plasmid were selected by screening for erythromycin sensitivity and loss of fluorescence marker phenotype. Integration of the SP32-X1 gene was confirmed by PCR analysis. One B. licheniformis integrant with the SP32-X1 gene inserted at two chromosomal loci was designated BT18062.

### Example 2. Construction of Bacillus licheniformis Strains BT18076, BT18074, and BT18068 Expressing Cutinase Encoded by Varying Gene Copy Numbers

Using conjugation donor strain BT18049 described in Example 1, plasmid pCLK015 was introduced by conjugation into a derivative of Bacillus licheniformis AEB1954 comprising five chromosomal target sites for insertion of the plasmid and deletions in the genes encoding alkaline protease (aprL), Glu-specific protease (mprL), bacillopeptidase F (bprAB), minor extracellular serine proteases (epr and vpr), secreted quality control protease (wprA) and intracellular serine protease (ispA). At each of the five chromosomal target sites of the B. licheniformis host is an expression cassette comprising a P3 promoter (SEQ ID NO: 14) followed by the crylllA mRNA stabilizer region (SEQ ID NO:18), a fluorescent marker gene (or a neo resistance marker gene) and an attB recombination site. One or more copies of the plasmid is inserted into the B. licheniformis chromosome by site-specific recombination between the attP sites on the plasmid, and attB sites at the target chromosomal loci. The plasmid(s) was then allowed to excise from the chromosome via homologous recombination by incubation at 34° C. in the absence of erythromycin selection. Integrants that had lost the plasmid were selected by screening for erythromycin sensitivity and loss of fluorescence- and neo-marker phenotypes. Integration of one or more copies of the SP32-X1 gene was confirmed by PCR analysis. One B. licheniformis integrant with the SP32-X1 gene inserted at three chromosomal loci was designated BT18064-2 and one B. licheniformis integrant with the SP32-X1 gene inserted at four chromosomal loci was designated BT18064-1. The remaining P3-marker gene expression cassettes of BT18064-1 and BT18064-2 were then removed using donor strain PP3713 and a similar integration procedure. The resulting strains BT18074 thus contains 4 copies of SP32-X1 and BT18076 contains 3 copies of SP32-X1. One B. licheniformis integrant with the SP32-X1 gene inserted at five chromosomal loci was designated BT18068.

### Example 3: Cutinase Yield Cannot be Increased by Increasing Cutinase Gene Copy Number

B. licheniformis strains BT18062, BT18076, BT18074, and BT18068 were tested with respect to cutinase productivity in fed-batch cultivations as described above. Cutinase production by the strains was compared using enzyme activity assay as described above. Relative total cutinase products are shown in Table 2. As can be seen from Table 2, compared to strain BT18062 (2 copies of the cutinase gene) the relative cutinase yield could not be increased by adding further cutinase gene copies, i.e. addition of a third copy, addition of two further copies, or addition of three further copies. Surprisingly, strains with more than 2 copies of the cutinase gene, i.e. strain BT18076 (3 copies), BT18074 (4 copies), and strain BT18068 (5 copies) resulted in cutinase yield lower and/or similar than the cutinase yield from the 2-copy strain BT18062, namely cutinase yield lowered by 1%, 5%, and 16%, respectively.

TABLE 2
Relative total cutinase product for B. licheniformis
strains expressing cutinase (N = 1). Yield samples
corresponds to yield after 120 hours of cultivation.

| | Number of cutinase | Relative |
|---|---|---|
| Strain | gene copies | product yield |
| BT18062 (2 copy) | 2 | 1.0 |
| BT18076 (3 copy) | 3 | 0.99 |
| BT18074 (4 copy) | 4 | 0.95 |
| BT18068 (5 copy) | 5 | 0.84 |

### Example 4. Construction of Bacillus licheniformis Strain BT14205 Expressing Cutinase from 5 Gene Copies with Heterologous Expression of yabJ-spoVG

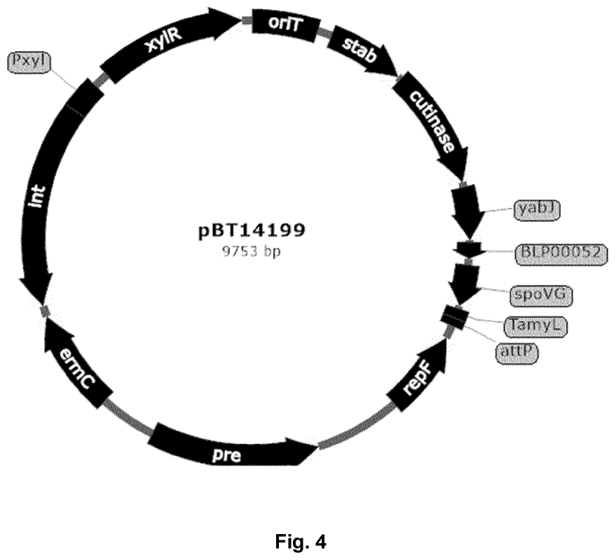

Plasmid pBT14199 was constructed for insertion of a gene encoding cutinase X1 with Bacillus licheniformis yabJ-spoVG in operon fusion into the genome of a Bacillus host using the site-specific recombinase-mediated method described above. The maps pBT14199 is shown in FIG. 4. Plasmid pBT14199 was introduced into conjugation donor strain Bacillus subtilis PP3724 by transformation, resulting in strain BT14199. Using conjugation donor strain BT14199, plasmid BT14199 was introduced by conjugation into a derivative of Bacillus licheniformis BT18064-1 comprising four copies of a Cutinase X1 gene and one chromosomal target site for insertion of the plasmid as described above. Integration of Cutinase-yabJ-spoVG was confirmed by PCR analysis. One B. licheniformis integrant with the Cutinase-yabJ-spoVG operon inserted at one chromosomal locus was designated BT14205.

### Example 5. Cutinase Production is Increased by Additional Copy of spoVG

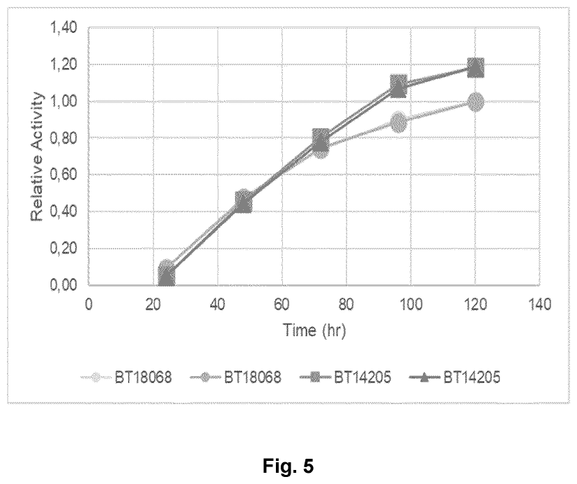

B. licheniformis strains BT18068 (5 copy cutinase gene) and BT14205 (5 copy cutinase gene+one additional spoVG copy) were tested with respect to cutinase X1 productivity in fed-batch cultivations as described above. Cutinase production by the strains was compared using an enzyme activity assay as described above. Relative total cutinase yield is shown in Table 3. As can be seen from Table 3 and FIG. 5, the strains with additional spoVG gene copy (BT14205) resulted in 18% increased cutinase yield compared to the strains lacking the additional spoVG gene copy (BT18068).

TABLE 3
Relative total cutinase yield for B. licheniformis
strains expressing cutinase (N = 2). Yield samples
corresponds to yield after 120 hours of cultivation.

| | Number of | | |
|---|---|---|---|
| | cutinase | Number of heterologous yabJ- | Relative |
| Strain | gene copies | BL00052-spoVG copies | product yield |
| BT18068 | 5 | 0 | 1.0 |
| BT14205 | 5 | 1 | 1.18 |

### Example 6: Cutinase Production is Increased Also in 2- and 3-Copy Cutinase Strains by Additional Copy of spoVG

Bacillus licheniformis strains expressing cutinase variant X2 from 2 or 3 gene copies and additional heterologous expression of yabJ-spoVG, were constructed. Using conjugation donor strains BT18093 and BT14255, plasmids pBT18093 and pBT14255 were introduced by conjugation into derivatives of Bacillus licheniformis AN1301 and AN1302. Using the PhIT integration procedure described above, Cutinase X2 copies and Cutinase X2-yabJ-spoVG copies were inserted in the chromosomes of AN1301 and AN1302, resulting in strains BT14265 (cutinase X2 in xylA- and lacA2-loci, and Cutinase X2-yabJ-spoVG in bglC locus) and BT14271 (Cutinase X2 in xylA locus and Cutinase X2-yabJ-spoVG in lacA2 locus).

As can be seen from Table 4, the strains with additional spoVG gene copy (BT14271 and BT14265) resulted in 9% and 13% increased cutinase yield compared to the strain lacking the additional spoVG gene copy (BT18093). It was shown in example 3 that yields from 2- and 3-copy Cutinase strains, lacking the extra spoVG copy, were comparable.

TABLE 4
Relative total cutinase yield for B. licheniformis
strains expressing cutinase. Yield samples corresponds
to yield after 120 hours of cultivation.

| | Number of | | |
|---|---|---|---|
| | cutinase | Number of heterologous yabJ- | Relative |
| Strain | gene copies | BL00052-spoVG copies | product yield |
| BT18093 | 2 | 0 | 1.0 |
| BT14271 | 2 | 1 | 1.09 |
| BT14265 | 3 | 1 | 1.13 |

### Example 7: 1-Copy of spoVG (SEQ ID NO:2; without YabJ) is Sufficient for Yield Increase

To show that spoVG, and not yabJ, is responsible for the observed yield increase, a series of 2-copy Cutinase strains with either additional spoVG and/or yabJ expression, were constructed. First, a Bacillus licheniformis 2-copy Cutinase strain was constructed by use of conjugation donor BT18089, a recipient strain with four chromosomal target sites for integration of PhIT plasmids (BKQ1867), followed by the PhIT process as described above. One B. licheniformis integrant with the SP32-X2 gene inserted at two chromosomal loci was designated BT14318. Additional copies of either yabJ, spoVG, or yabJ-spoVG, were inserted in BT14318 by use of conjugation donors BT14317 (yabJ), BT14316 (spoVG), and BT14285 (yabJ-spoVG). The resulting set of 2-copy Cutinase X2 strains with various levels of yabJ and spoVG expression are shown in Table 5 below. As can be seen from Table 5, only strains with additional spoVG gene copies (BT14222-BT14225) results in increased cutinase yield compared to the strain with only additional yabJ gene (normalized to gene copy number).

TABLE 5
Relative total cutinase yield for B. licheniformis
strains expressing cutinase. Yield samples corresponds
to yield after 120 hours of cultivation.

| | | Number of | | |
|---|---|---|---|---|
| | Number of | heterologous | Number of | |
| | heterologous | yabJ-spoVG | heterologous | Relative |
| Strain | yabJ copies | copies | spoVG copies | product yield |
| BT14327 | 1 | 0 | 0 | 1 |
| BT14323 | 0 | 1 | 0 | 1.35 |
| BT14325 | 0 | 0 | 1 | 1.36 |
| BT14326 | 2 | 0 | 0 | 1 |
| BT14322 | 0 | 2 | 0 | 1.26 |
| BT14324 | 0 | 0 | 2 | 1.36 |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

The invention is further defined by the following numbered paragraphs:
1. A recombinant bacterial host cell comprising in its genome at least one first heterologous promoter operably linked to at least one first polynucleotide encoding a stage 5 sporulation protein G (SpoVG) polypeptide.
2. The host cell according to claim 1, wherein the SpoVG polypeptide is a SpoVG fragment or a SpoVG variant.
3. The host cell according to any of the preceding paragraphs, further comprising in its genome at least one second polynucleotide encoding at least one polypeptide of interest.
4. The host cell according to paragraph 3, wherein the polypeptide of interest is endogenous to the host cell.
5. The host cell according to paragraph 3, wherein the polypeptide of interest is exogenous to the host cell.
6. The host cell according to any of paragraphs 3 to 5, wherein the second polynucleotide is operably linked a heterologous promoter, preferably to the first heterologous promoter.
7. The host cell according to paragraph 6, wherein the second polynucleotide is located downstream at the 3′ end of the first polynucleotide.
8. The host cell according to paragraph 6, wherein the second polynucleotide is located upstream at the 5′ end of the first polynucleotide.
9. The host cell according to any of paragraphs 3 to 5, wherein the second polynucleotide is operably linked to a second promoter, preferably the second promoter is a heterologous promoter.
10. The host cell according to paragraph 9, wherein the first heterologous promoter comprises, or consists of a nucleic acid sequence having a sequence identity of at least 80%, e.g., at least 85%, at least 90%, at least 91%, at least 92%, at least 93%, at least 94%, at least 95%, at least 96%, at least 97%, at least 98%, at least 99%, or 100% to the nucleic acid sequence of the second promoter.
11. The host cell according to any of paragraphs 9 to 10, wherein the nucleic acid sequence of the first heterologous promoter and the nucleic acid sequence of the second promoter are identical.
12. The host cell according to any of the preceding paragraphs, wherein the first heterologous promoter comprises or consists of a nucleic acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 14.
13. The host cell according to any of paragraphs 9 to 12, wherein the second promoter comprises or consists of a nucleic acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 14.
14. The host cell according to any of paragraphs 3 to 14, wherein the at least one polypeptide of interest is secreted.
15. The host cell according to any of paragraphs 3 to 14, wherein the at least one polypeptide of interest is not secreted.
16. The host cell according to paragraph 15, wherein the at least one polypeptide of interest comprises, or consists of an asparaginase.
17. The bacterial host cell according to any of the preceding paragraphs, wherein the host cell comprises at least two copies of the first heterologous promoter operably linked to the first polynucleotide, such as at least two copies, at least three copies, at least four copies, at least five copies, or at least six copies.
18. The bacterial host cell according to any of the preceding paragraphs, wherein the SpoVG polypeptide, SpoVG fragment, or SpoVG variant is endogenous to the host cell.
19. The bacterial host cell according to any of paragraphs 1 to 17, wherein the SpoVG polypeptide, SpoVG fragment, or SpoVG variant is exogenous to the host cell.
20. The bacterial host cell according to any of the preceding paragraphs, wherein the SpoVG polypeptide, SpoVG fragment, or SpoVG variant comprises, or consists of an amino acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 2 or SEQ ID NO: 20
21. The bacterial host cell according to any of the preceding paragraphs, wherein the first polynucleotide encoding the SpoVG polypeptide, SpoVG fragment, or SpoVG variant comprises, or consists of a nucleic acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 1, SEQ ID NO: 7, or SEQ ID NO: 19.
22. The bacterial host cell according to any of the preceding paragraphs, wherein the first polynucleotide encodes a stage 5 sporulation protein G (SpoVG) polypeptide, a SpoVG fragment or a SpoVG variant, AND a YabJ polypeptide, a YabJ fragment, or a YabJ variant.
23. The bacterial host cell according to paragraph 22, wherein the SpoVG polypeptide, SpoVG fragment, or SpoVG variant is translated into a polypeptide, fragment or variant not sharing the same polypeptide chain with the YabJ polypeptide, YabJ fragment, or YabJ variant.
24. The host cell according to any of paragraphs 22 to 23, wherein the polynucleotide encoding the YabJ polypeptide, YabJ fragment, or YabJ variant is located upstream at the 5′ end of the polynucleotide encoding the SpoVG polypeptide, SpoVG fragment, or SpoVG variant.
25. The host cell according to any of paragraphs 22 to 23, wherein the polynucleotide encoding the YabJ polypeptide, YabJ fragment, or YabJ variant is located downstream at the 3′ end of the polynucleotide encoding the SpoVG polypeptide, SpoVG fragment, or SpoVG variant.
26. The host cell according to any of paragraphs 22 to 25, wherein the YabJ polypeptide, YabJ fragment, or YabJ variant comprises, or consists of an amino acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 6.
27. The host cell according to any of paragraphs 22 to 26, wherein the polynucleotide encoding the YabJ polypeptide, YabJ fragment, or YabJ variant comprises, or consists of a nucleic acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 5 or SEQ ID NO: 10.
28. The host cell according to any of paragraphs 22 to 27, wherein the first polynucleotide comprises or consists of a nucleic acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 8 or SEQ ID NO: 9.
29. The host cell according to any of the preceding paragraphs, wherein the first polynucleotide and/or the second polynucleotide is/are operably linked in translational fusion with a third polynucleotide encoding a signal peptide.
30. The host cell according to paragraph 29, wherein the third polynucleotide is operably linked in translational fusion with the first and/or the second polypeptide.
31. The host cell according to any of paragraphs 29 to 30, wherein the third polynucleotide encodes a signal peptide comprising, or consisting of an amino acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 16.
32. The host cell according to any of paragraphs 3 to 31, comprising in its genome a polynucleotide comprising from its 5′ end to its 3′ end:
   a) the first heterologous promoter,
   b) the first polynucleotide encoding the SpoVG polypeptide, and optionally the YabJ polypeptide, operably linked to the first heterologous promoter, and
   c) the second polynucleotide encoding the polypeptide of interest.
33. The host cell according to any of paragraphs 3 to 31, comprising in its genome a polynucleotide comprising from its 5′ end to its 3′ end:
   a) the first heterologous promoter,
   b) the second polynucleotide encoding the polypeptide of interest, operably linked to the first heterologous promoter, and
   c) the first polynucleotide encoding the SpoVG polypeptide, and optionally the YabJ polypeptide.
34. The host cell according to paragraph 33, wherein the polynucleotide encoding the elements a), b) and c) comprises, or consists of a polynucleotide having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 13 or SEQ ID NO: 12.
35. The host cell according to any of paragraphs 3 to 31, comprising in its genome a polynucleotide comprising from its 5′ end to its 3′ end:
   a) the first polynucleotide encoding the SpoVG polypeptide, and optionally the YabJ polypeptide, and
   b) the second polynucleotide encoding the polypeptide of interest.
36. The host cell according to any of paragraphs 3 to 31, comprising in its genome a polynucleotide comprising from its 5′ end to its 3′ end:
   a) the second polynucleotide encoding the polypeptide of interest, and
   b) the first polynucleotide encoding the SpoVG polypeptide, and optionally the YabJ polypeptide.
37. The host cell according to paragraph 36, wherein the polynucleotide encoding the elements a) and b) comprises, or consists of a polynucleotide having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 11 or SEQ ID NO: 12.
38. The host cell according to any of paragraphs 3 to 31, comprising in its genome a polynucleotide comprising from its 5′ end to its 3′ end:
   a) the first heterologous promoter, and
   b) the first polynucleotide encoding the SpoVG polypeptide, and optionally the YabJ polypeptide, operably linked to the first heterologous promoter. 39. The host cell according to paragraph 38, wherein the polynucleotide encoding the elements a) and b) comprises, or consists of a polynucleotide having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 21, SEQ ID NO: 22, or SEQ ID NO: 23.
40. The host cell according to any of paragraphs 3 to 39, wherein the host cell comprises in its genome at least two copies of the second polynucleotide, such as at least two copies, at least three copies, at least four copies, at least five copies, or at least six copies.
41. The host cell according to any of the preceding paragraphs, wherein the first heterologous promoter operably linked to the first polynucleotide is endogenous to the host cell.
42. The host cell according to any of paragraphs 1 to 40, wherein the first heterologous promoter operably linked to the first polynucleotide is exogenous to the host cell.
43. The host cell according to any of the preceding paragraphs, wherein total mRNA of the first polynucleotide encoding the SpoVG polypeptide, the SpoVG fragment, and/or the SpoVG variant is increased relative to total mRNA of the native SpoVG gene SpoVG polypeptide, the SpoVG fragment, and/or the SpoVG variant in a parent host cell which does not comprise the first polynucleotide operably linked to the first heterologous promoter, when cultivated under identical conditions.
44. The host cell according to paragraph 43, wherein the parent host cell is otherwise isogenic to the host cell according to any of paragraphs 1 to 42.
45. The host cell according to any of paragraphs 43 to 44, wherein total mRNA of the first polynucleotide encoding the SpoVG polypeptide, the SpoVG fragment, and/or the SpoVG variant is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, at least 215%, at least 220%, at least 225%, at least 230%, at least 235%, at least 240%, at least 245%, at least 250%, at least 255%, at least 260%, at least 265%, at least 270%, at least 275%, at least 280%, at least 285%, at least 290%, at least 295%, at least 300%, at least 305%, at least 310%, at least 315%, at least 320%, at least 325%, at least 330%, at least 335%, at least 340%, at least 345%, at least 350%, at least 355%, at least 360%, at least 365%, at least 370%, at least 375%, at least 380%, at least 385%, at least 390%, at least 395%, or at least 400%.
46. The host cell according to any of the preceding paragraphs, wherein expression of the SpoVG polypeptide, the SpoVG fragment, and/or the SpoVG variant is increased relative to the expression of the native SpoVG polypeptide, the SpoVG fragment, and/or the SpoVG variant in a parent host cell which does not comprise the first polynucleotide operably linked to the first heterologous promoter, when cultivated under identical conditions.
47. The host cell according to paragraph 46, wherein the parent host cell is otherwise isogenic to the host cell according to any of paragraphs 1 to 42.
48. The host cell according to any of paragraphs 43 to 47, wherein expression of the SpoVG polypeptide, the SpoVG fragment, and/or the SpoVG variant is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, at least 215%, at least 220%, at least 225%, at least 230%, at least 235%, at least 240%, at least 245%, at least 250%, at least 255%, at least 260%, at least 265%, at least 270%, at least 275%, at least 280%, at least 285%, at least 290%, at least 295%, at least 300%, at least 305%, at least 310%, at least 315%, at least 320%, at least 325%, at least 330%, at least 335%, at least 340%, at least 345%, at least 350%, at least 355%, at least 360%, at least 365%, at least 370%, at least 375%, at least 380%, at least 385%, at least 390%, at least 395%, or at least 400%.
49. The bacterial host cell according to any of the preceding paragraphs, wherein the host cell is a Gram-negative bacteria selected from the group consisting of Campylobacter, E. coli, Flavobacterium, Fusobacterium, Helicobacter, Ilyobacter, Neisseria, Pseudomonas, Salmonella, and Ureaplasma cells, or wherein the host cell is a Gram-positive cell selected from the group consisting of Bacillus, Clostridium, Enterococcus, Geobacillus, Lactobacillus, Lactococcus, Oceanobacillus, Staphylococcus, Streptococcus, or Streptomyces cells, such as Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus thuringiensis, Streptococcus equisimilis, Streptococcus pyogenes, Streptococcus uberis, and Streptococcus equi subsp. Zooepidemicus, Streptomyces achromogenes, Streptomyces avermitilis, Streptomyces coelicolor, Streptomyces griseus, and Streptomyces lividans cells, preferably the host cell is a Bacillus cell, most preferably a Bacillus subtilis or a Bacillus licheniformis cell.
50. The host cell according to any of the preceding paragraphs, wherein the host cell is a Bacillus cell.
51. The host cell according to any of the preceding paragraphs, wherein the host cell is a Bacillus subtilis cell.
52. The host cell according to any of the preceding paragraphs, wherein the host cell is a Bacillus licheniformis cell.
53. The host cell according to any of the preceding paragraphs, wherein the one or more polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deamidase, deoxyribonuclease, endoglucanase, esterase, alpha-galactosidase, beta-galactosidase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase; even more preferably the one or more polypeptide of interest comprises a cutinase.
54. The host cell according to any of the preceding paragraphs, wherein the polypeptide of interest comprises a cutinase.
55. The host cell according to any of the preceding paragraphs, wherein the one or more polypeptide of interest is a cutinase, such as a cutinase which comprises, consists essentially of, or consists of the mature polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 4.
56. The host cell according to any of the preceding paragraphs, which is isolated.
57. The host cell according to any of the preceding paragraphs, which is purified.
58. A method for producing one or more polypeptide of interest, the method comprising:
   a) providing a bacterial host cell according to any one of paragraphs 1 to 57,
   b) cultivating said host cell under conditions conducive for expression of the one or more polypeptide of interest; and,
   c) optionally recovering the one or more polypeptide of interest.
59. A nucleic acid construct comprising at least one first heterologous promoter operably linked to at least one first polynucleotide encoding a stage 5 sporulation protein G (SpoVG) polypeptide.
60. The nucleic acid construct according to paragraph 59, wherein the SpoVG polypeptide is a SpoVG fragment or a SpoVG variant.
61. The nucleic acid construct according to any of paragraphs 59 to 60, further comprising at least one second polynucleotide encoding at least one polypeptide of interest.
62. The nucleic acid construct according to paragraph 61, wherein the second polynucleotide is operably linked to the first heterologous promoter.
63. The nucleic acid construct according to paragraph 62, wherein the second polynucleotide is located downstream at the 3′ end of the first polynucleotide.
64. The nucleic acid construct according to paragraph 62, wherein the second polynucleotide is located upstream at the 5′ end of the first polynucleotide.
65. The nucleic acid construct according to any of paragraphs 61 to 64, wherein the second polynucleotide is operably linked to a second promoter, preferably the second promoter is a heterologous promoter.
66. The nucleic acid construct according to paragraph 65, wherein the first heterologous promoter comprises, or consists of a nucleic acid sequence having a sequence identity of at least 80%, e.g., at least 85%, at least 90%, at least 91%, at least 92%, at least 93%, at least 94%, at least 95%, at least 96%, at least 97%, at least 98%, at least 99%, or 100% to the nucleic acid sequence of the second promoter.
67. The nucleic acid construct according to any of paragraphs 65 to 66, wherein the nucleic acid sequence of the first heterologous promoter and the nucleic acid sequence of the second promoter are identical.
68. The nucleic acid construct according to any of paragraphs 59 to 67, wherein the first heterologous promoter comprises or consists of a nucleic acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 14.
69. The nucleic acid construct according to any of paragraphs 65 to 68, wherein the second promoter comprises or consists of a nucleic acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 14.
70. The nucleic acid construct according to any of paragraphs 59 to 69, wherein the SpoVG polypeptide, SpoVG fragment, or SpoVG variant comprises, or consists of an amino acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 2 or SEQ ID NO: 20.
71. The nucleic acid construct according to any of paragraphs 59 to 70, wherein the first polynucleotide encoding the SpoVG polypeptide, SpoVG fragment, or SpoVG variant comprises, or consists of a nucleic acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 1, SEQ ID NO: 7, or SEQ ID NO: 19.
72. The nucleic acid construct according to any of paragraphs 59 to 71, wherein the first polynucleotide encodes a stage 5 sporulation protein G (SpoVG) polypeptide, a SpoVG fragment or a SpoVG variant, AND a YabJ polypeptide, a YabJ fragment, or a YabJ variant.
73. The nucleic acid construct according to paragraph 72, wherein the SpoVG polypeptide, SpoVG fragment, or SpoVG variant is translated into a polypeptide, fragment or variant not sharing the same polypeptide chain with the YabJ polypeptide, YabJ fragment, or YabJ variant.
74. The nucleic acid construct according to any of paragraphs 72 to 73, wherein the polynucleotide encoding the YabJ polypeptide, YabJ fragment, or YabJ variant is located upstream at the 5′ end of the polynucleotide encoding the SpoVG polypeptide, SpoVG fragment, or SpoVG variant.
75. The nucleic acid construct according to any of paragraphs 72 to 73, wherein the polynucleotide encoding the YabJ polypeptide, YabJ fragment, or YabJ variant is located downstream at the 3′ end of the polynucleotide encoding the SpoVG polypeptide, SpoVG fragment, or SpoVG variant.
76. The nucleic acid construct according to any of paragraphs 72 to 75, wherein the YabJ polypeptide, YabJ fragment, or YabJ variant comprises, or consists of an amino acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 6.
77. The nucleic acid construct according to any of paragraphs 72 to 76, wherein the polynucleotide encoding the YabJ polypeptide, YabJ fragment, orYabJ variant comprises, or consists of a nucleic acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 5 or SEQ ID NO: 10.
78. The nucleic acid construct according to any of paragraphs 72 to 76, wherein the first polynucleotide comprises or consists of a nucleic acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 8 or SEQ ID NO: 9.
79. The nucleic acid construct according to any of paragraphs 61 to 78, wherein the first polynucleotide and/or the second polynucleotide is/are operably linked in translational fusion with a third polynucleotide encoding a signal peptide.
80. The nucleic acid construct according to paragraph 79, wherein the third polynucleotide is operably linked in translational fusion with the first and/or the second polypeptide.
81. The nucleic acid construct according to any of paragraphs 79 to 80, wherein the third polynucleotide encodes a signal peptide comprising, or consisting of an amino acid sequence having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 16.
82. The nucleic acid construct according to any of paragraphs 61 to 81, comprising a polynucleotide comprising from its 5′ end to its 3′ end:
   a) the first heterologous promoter,
   b) the first polynucleotide encoding the SpoVG polypeptide, and optionally the YabJ polypeptide, operably linked to the first heterologous promoter, and
   c) the second polynucleotide encoding the polypeptide of interest.
83. The nucleic acid construct according to any of paragraphs 61 to 81, comprising a polynucleotide comprising from its 5′ end to its 3′ end:
   a) the first heterologous promoter,
   b) the second polynucleotide encoding the polypeptide of interest, operably linked to the first heterologous promoter, and
   c) the first polynucleotide encoding the SpoVG polypeptide, and optionally the YabJ polypeptide.
84. The nucleic acid construct according to paragraph 83, wherein the polynucleotide encoding the elements a), b) and c) comprises, or consists of a polynucleotide having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 13 or SEQ ID NO: 12.
85. The nucleic acid construct according to any of paragraphs 61 to 81, comprising a polynucleotide comprising from its 5′ end to its 3′ end:
   a) the first polynucleotide encoding the SpoVG polypeptide, and optionally the YabJ polypeptide, and
   b) the second polynucleotide encoding the polypeptide of interest.
86. The nucleic acid construct according to any of paragraphs 61 to 81, comprising a polynucleotide comprising from its 5′ end to its 3′ end:
   a) the second polynucleotide encoding the polypeptide of interest, and
   b) the first polynucleotide encoding the SpoVG polypeptide, and optionally the YabJ polypeptide.
87. The nucleic acid construct according to paragraph 86, wherein the polynucleotide encoding the elements a) and b) comprises, or consists of a polynucleotide having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 11 or SEQ ID NO: 12.
88. The nucleic acid construct according to any of paragraphs 61 to 81, comprising a polynucleotide comprising from its 5′ end to its 3′ end:
   a) the first heterologous promoter, and
   b) the first polynucleotide encoding the SpoVG polypeptide, and optionally the YabJ polypeptide, operably linked to the first heterologous promoter.
89. The nucleic acid construct according to paragraph 88, wherein the polynucleotide encoding the elements a) and b) comprises, or consists of a polynucleotide having a sequence identity of least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the nucleic acid sequence of SEQ ID NO: 21, SEQ ID NO: 22, or SEQ ID NO: 23.
90. The nucleic acid construct according to any of paragraphs 61 to 89, wherein the one or more polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deamidase, deoxyribonuclease, endoglucanase, esterase, alpha-galactosidase, beta-galactosidase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase; even more preferably the one or more polypeptide of interest comprises a cutinase.
91. The nucleic acid construct according to any of paragraphs 61 to 90, wherein the polypeptide of interest comprises a cutinase.
92. The nucleic acid construct according to any of paragraphs 61 to 91, wherein the one or more polypeptide of interest is a cutinase, such as a cutinase which comprises, consists essentially of, or consists of the mature polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 4.
93. An expression vector comprising a nucleic acid construct according to any of paragraphs 59 to 92.
94. A recombinant bacterial host cell comprising in its genome a nucleic acid construct according to any of paragraphs 59 to 92, or an expression vector according to paragraph 93, preferably wherein the host cell is a Bacillus host cell, more preferably the host cell is a Bacillus subtilis host cell or a Bacillus licheniformis host cell.
95. A method for production of a recombinant bacterial host cell with increased yield of a polypeptide of interest, the method comprising:
   a) providing a parent bacterial host cell comprising in its genome a native polynucleotide encoding a SpoVG polypeptide, SpoVG variant, or SpoVG fragment,
   b) introducing into said parent bacterial host cell a first polynucleotide encoding the SpoVG polypeptide, fragment or variant, and
   c) introducing into said parent bacterial host cell a second polynucleotide encoding the polypeptide of interest,
   wherein step c) is carried out prior to step b), in parallel to step b), or after step b), and wherein the recombinant bacterial host cell comprises increased expression of the SpoVG polypeptide, fragment or variant relative to the parent bacterial host cell when cultivated under identical conditions.
96. The method according to paragraph 95, wherein a polynucleotide construct according to any of paragraphs 59 to 92, and/or an expression vector according to paragraph 93 is introduced into the host cell during step b) and/or step c).
97. A method for production of a recombinant bacterial host cell with increased yield of a polypeptide of interest, the method comprising:
   a) providing a parent bacterial host cell comprising in its genome a native polynucleotide encoding a SpoVG polypeptide, SpoVG variant, or SpoVG fragment, and a second polynucleotide encoding a polypeptide of interest, and
   b) introducing into said parent bacterial host cell a first polynucleotide encoding the SpoVG polypeptide, fragment or variant, and wherein the recombinant bacterial host cell comprises increased expression of the SpoVG polypeptide, fragment or variant relative to the parent bacterial host cell when cultivated under identical conditions.
98. The method according to paragraph 97, wherein a polynucleotide construct according to any of paragraphs 59 to 92, and/or an expression vector according to paragraph 93 is introduced into the host cell during step b).

1: A recombinant bacterial host cell comprising in its genome at least one first heterologous promoter operably linked to at least one first polynucleotide encoding a stage 5 sporulation protein G (SpoVG) polypeptide, a SpoVG fragment, or a SpoVG variant.

2: The host cell according to claim 1, further comprising in its genome at least one second polynucleotide encoding at least one polypeptide of interest.

3: The host cell according to claim 2, wherein the second polynucleotide is operably linked to a heterologous promoter.

4: The host cell according to claim 1, wherein the host cell comprises at least two copies of the first heterologous promoter operably linked to the first polynucleotide.

5: The host cell according to claim 1, wherein the SpoVG polypeptide, SpoVG fragment, or SpoVG variant comprises, or consists of an amino acid sequence having a sequence identity of least 60% to the amino acid sequence of SEQ ID NO: 2 or SEQ ID NO: 20.

6: The host cell according to claim 1, wherein expression of the SpoVG polypeptide, the SpoVG fragment, and/or the SpoVG variant is increased relative to the expression of the native SpoVG polypeptide, the SpoVG fragment, and/or the SpoVG variant in a parent host cell which does not comprise the first polynucleotide operably linked to the first heterologous promoter, when cultivated under identical conditions.

7: The host cell according to claim 1, wherein the host cell is a Gram-negative bacteria selected from the group consisting of Campylobacter, E. coli, Flavobacterium, Fusobacterium, Helicobacter, Ilyobacter, Neisseria, Pseudomonas, Salmonella, and Ureaplasma cells, or wherein the host cell is a Gram-positive cell selected from the group consisting of Bacillus, Clostridium, Enterococcus, Geobacillus, Lactobacillus, Lactococcus, Oceanobacillus, Staphylococcus, Streptococcus, or Streptomyces cells, such as Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus thuringiensis, Streptococcus equisimilis, Streptococcus pyogenes, Streptococcus uberis, and Streptococcus equi subsp. Zooepidemicus, Streptomyces achromogenes, Streptomyces avermitilis, Streptomyces coelicolor, Streptomyces griseus, and Streptomyces lividans cells.

8: The host cell according to claim 1, wherein the one or more polypeptide of interest comprises an enzyme.

9: The host cell according to claim 1, wherein the one or more polypeptide of interest is a cutinase.

10: A method for producing one or more polypeptides of interest, the method comprising:
a) providing a bacterial host cell according to claim 1, and
b) cultivating said host cell under conditions conducive for expression of the one or more polypeptides of interest.

11: A nucleic acid construct comprising at least one first heterologous promoter operably linked to at least one first polynucleotide encoding a stage 5 sporulation protein G (SpoVG) polypeptide, a SpoVG fragment, or a SpoVG variant.

12: An expression vector comprising a nucleic acid construct according to claim 11.

13: A method for production of a recombinant bacterial host cell with increased expression of a polypeptide of interest, the method comprising:
a) providing a parent bacterial host cell comprising in its genome a native polynucleotide encoding a SpoVG polypeptide, SpoVG variant, or SpoVG fragment, and a second polynucleotide encoding a polypeptide of interest, and
b) introducing into said parent bacterial host cell a first polynucleotide encoding the SpoVG polypeptide, fragment or variant,
wherein the recombinant bacterial host cell comprises increased expression of the polypeptide of interest and the SpoVG polypeptide, fragment or variant relative to the parent bacterial host cell when cultivated under identical conditions.

14. (canceled)

15: The host cell according to claim 3, wherein the second polynucleotide is operably linked to the first heterologous promoter.

16: The host cell according to claim 1, wherein the host cell is a Bacillus subtilis or a Bacillus licheniformis cell.

17: The host cell according to claim 1, wherein the one or more polypeptide of interest comprises an enzyme selected an aminopeptidase, amylase, carbohydrase, carboxypeptidase, catalase, cellobiohydrolase, cellulase, chitinase, cutinase, cyclodextrin glycosyltransferase, deamidase, deoxyribonuclease, endoglucanase, esterase, alpha-galactosidase, beta-galactosidase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase and beta-xylosidase.

18: The host cell according to claim 1, wherein the one or more polypeptide of interest is a cutinase which comprises, consists essentially of, or consists of the mature polypeptide having a sequence identity of at least 60% to the amino acid sequence of SEQ ID NO: 4.

19: The method of claim 10, said method further comprising:
c) recovering the one or more polypeptides of interest.

20: The method according to claim 13, wherein a nucleic acid construct comprising at least one first heterologous promoter operably linked to at least one first polynucleotide encoding a stage 5 sporulation protein G (SpoVG) polypeptide, a SpoVG fragment, or a SpoVG variant is introduced into the host cell during step b).
